(19) United States
(12) Patent Application Publication
(10) Pub. No.: US 2015/0283066 A1
(43) Pub. Date: Oct. 8, 2015

(54) VAGINAL DANAZOL COMBINED WITH NON STEROIDAL ANTI INFLAMMATORY DRUGS (NSAIDS) COMPOSITIONS

(71) Applicant: MEDIGLOBE LTD, Misgav (IL)

(72) Inventor: Daniel Katz, Misgav (IL)

(21) Appl. No.: 14/344,939

(22) PCT Filed: Sep. 12, 2012
(86) PCT No.: PCT/IL12/00338

§ 371 Date: Oct. 8, 2014

(30) Foreign Application Priority Data
Sep. 18, 2011 (IL) 215224

(51) Int. Cl. A61K 9/00; A61K 31/196; A61K 9/20; A61K 31/5415; A61K 31/405; A61K 9/46; A61K 31/192

(52) U.S. Cl. CPC A61K 9/0034; A61K 9/0007; A61K 31/196; A61K 31/192; A61K 31/5415; A61K 31/405; A61K 9/2027; A61K 9/2059; A61K 9/2013; A61K 9/2009; A61K 9/2018

(57) ABSTRACT

The present invention discloses a pharmaceutical composition useful for treating a gynecological condition comprising (a) an active ingredient selected from a group consisting of at least one non-steroidal anti-inflammatory drug (NSAID), danazol and a combination thereof, (b) at least one pharmaceutically acceptable carrier or excipient. In a core aspect of the invention that the composition is adapted to be vaginally administrable further wherein said composition is formed in an immediate release form. Kits and methods using the aforementioned composition are also disclosed.

### FIELD OF THE INVENTION

This invention is directed towards pharmacological Non Steroidal Anti Inflammatory Drugs (NSAIDs) combined with danazol compositions and methods for use in treatment of gynecological disorders. Specifically, the present invention pertains to vaginally administrable pharmaceutical compositions comprising NSAID, danazol or a combination thereof for the treatment of endometriosis, adenomyosis, menorrhagia, dysmenorrheal and menstruation pains.

### BACKGROUND

Danazol, an androgen derivative which suppresses the pituitary-ovarian axis by inhibiting the release of GnRH, is well known in the art.

Danazol (Danocrine), a testosterone agonist, is a suppressive steroid with some androgenic activity. It inhibits the growth of endometriosis but its use remains limited as it may cause hirsutism and voice changes. Danazol is known as a drug for the oral treatment of pelvic endometriosis.

Danazol is a derivative of the synthetic steroid ethisterone, a modified testosterone, also known as 17alpha-ethinyl testosterone. Recent studies suggest that danazol, which acts as an inhibitor at the anterior lobe of pituitary, inhibits the secretion of hormones from the hypothalamus or pituitary or from ovary upon its oral administration and thereby exhibits therapeutic effect on endometriosis. Thus, danazol having such a functional mechanism as above has been used as a systemic therapeutic agent for oral administration.

Although effective for endometriosis, the use of danazol, as a systemic therapeutic agent, is limited by its masculinizing side-effects, including weight gain, acne, oily skin and hair, bloating, fluid retention, voice changes, increase in body hair, decreased breast size, decreased libido and enlargement of the clitoris (Selak et al, 2007; Flower et al, 2007; Luisi et al, 2009). Long-term use is associated with a small risk of developing liver tumours and a theoretical risk of developing heart disease (Selak et al, 2007).

Moreover, bioavailability studies indicate that blood levels do not increase proportionally with the increase in the administered dose. When the dose of Danazol is doubled the increase in plasma levels is only about 35% to 40% (FDA approved information for healthcare professionals and patients).

Vaginal application of danazol has reduced side effects as compared to systemic administration of the drug. None of the side effects such as the increase of body weight, the growth of blackhead, (transient) hepatic insufficiency or the like which have often been observed in the treatment with the oral administration of danazol are accompanied with the topical administration of danazol. For example, EP0501056A1 discloses vaginal administration of danazol as a therapeutic agent of endometriosis but does not provide a solution to treating or preventing pain and inflammation associated with such diseases.

Pelvic pain, unassociated with menses, may restrict afflicted women to limited participation in athletic and other physical activities. Through dyspareunia, they suffer not only the pain and often-missed orgasmic fulfillment, but perhaps even marital discord, separation, or infertility. Through relative infertility, those women may suffer further reductions in self-esteem that might result in further personal, physical, and economic cost. Often, coital events or pelvic exams produce pelvic aching for hours or even days thereafter. The peri-menstrual pain experienced by afflicted women may be relieved in part by non-steroidal anti-inflammatory drugs (NSAID's) given orally, as the U.S. Pat. No. 9,917,523 declares.

Heavy menstrual bleeding (HMB) is an important cause of ill health in premenopausal women. Surgery is sometimes used as a treatment and a range of medical therapies are also available. Nonsteroidal anti-inflammatory drugs reduce prostaglandin levels which are elevated in women with excessive menstrual bleeding and also may have a beneficial effect on dysmenorrhoea.

Researches have shown that non-steroidal anti-inflammatory drugs (NSAIDs) and tranexamic acid reduce menstrual blood loss by 20-60%, and the effectiveness of a hormonal intrauterine system (IUS) is comparable with that of endometrial ablation or hysterectomy. (Department of Obstetrics and Gynecology, University of Helsinki, Finland)

Controlled release pharmaceuticals have become very important in the treatment of many medical conditions. Such controlled release formulations have in fact, been found to be desirable in treating many chronic conditions, such as chronic pain, that would otherwise require inconvenient multiple daily doses. Additionally, controlled release dosage forms tend to maintain more consistent blood serum levels with less fluctuation, and thus may reduce undesirable side effects. However, because of the nature of certain types of drugs, often, it is desirable to modify or carry drugs in specific ways for even immediate release drugs. Thus, improved controlled release formulations and immediate release formulations that provide certain advantages continue to be sought.

Thus it would be very useful to have a treatment or therapy that effectively improves or cures menorrhagia, dysmenorrheal and heavy and prolonged menstruation bleeding and pain.

### SUMMARY OF THE INVENTION

It is an object of the present invention to disclose a pharmaceutical composition useful for treating a gynecological condition comprising (a) an active ingredient selected from a group consisting of at least one non-steroidal anti-inflammatory drug (NSAID), danazol and a combination thereof, (b) at least one pharmaceutically acceptable carrier or excipient, wherein the composition is adapted to be vaginally administrable further wherein the composition is in an immediate release form.

It is a further object of the present invention to disclose the pharmaceutical composition, wherein the gynecological condition is selected from a group consisting of endometriosis, adenomyosis, mennorhagia, dysmenorrheal, menstruation pains and any combination thereof.

It is a further object of the present invention to disclose the pharmaceutical composition, wherein the composition is adapted to reduce menstrual blood loss by at least about 10%.

It is a further object of the present invention to disclose the pharmaceutical composition, wherein the composition is formulated in a form selected from a group consisting of a solid dosage form, foam and a mousse.

It is a further object of the present invention to disclose the pharmaceutical composition, wherein the composition is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

It is a further object of the present invention to disclose the pharmaceutical composition, wherein the carrier or excipient is selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

It is a further object of the present invention to disclose the pharmaceutical composition, wherein the composition comprises between about 5 to about 12 wt. % effervescent excipient.

It is a further object of the present invention to disclose the pharmaceutical composition, wherein the at least one Thin Film type or PharmFilm type or mucoadhesive type excipient is selected from a group consisting of: hydroxypropyl methyl cellulose (HPMC) K4M, Carbopol 934, hyaluronic acid, Gelatin, Mannitol, Citric acid, Chitosan, Sodium Alignate, Cyclodextrin and combination thereof.

It is a further object of the present invention to disclose the pharmaceutical composition, wherein the at least one FDT type excipient is selected from a group consisting of: Mannitol, Lactose, Erythritol, Xylitol, Glucose, Sucrose, Sorbitol, Maltitol, Trehalose, Maltose and mixtures thereof.

It is a further object of the present invention to disclose the pharmaceutical composition, wherein the bioadhesive type agent is selected from a group consisting of Carbopol 971, hyaluronic acid, HPMC, hydrophilic polymers such as polyox, PEG (Polyethylene glycol), hydrogels, cellulose derivatives such as, sodium alginate, carboxymethylcellulose, hydroxymethylcellulose, methylcellulose, hydroxypropyl methyl cellulose (HPMC) K4M, Carbopol 934, Gelatin, Mannitol, Citric acid, Chitosan, Sodium Alignate, Cyclodextrin and any mixture thereof.

It is a further object of the present invention to disclose the pharmaceutical composition, wherein the probiotic agent is selected from a group consisting of lactic-acid bacteria such as Lactobacillus rhamnosus, bifidobacterium, streptococcus, factors that stimulate the growth of protective organisms, enzymes, vitamins and cellular factors and mixtures thereof.

It is a further object of the present invention to disclose the pharmaceutical composition, wherein the acidifying agent is selected from a group consisting of boric acid, acetic acid, glacial acetic acid, fumaric acid, diluted hydrochloric acid, citric acid, lactic acid, malic acid, nitric acid, phosphoric acid, sulfuric acid, tartaric acid, acidic buffer and mild organic acids approved for pharmaceutical applications and any combination thereof.

It is a further object of the present invention to disclose the pharmaceutical composition, wherein the surfactant is selected from a group consisting of: a hydrophilic, a non hydrophilic, a polymeric, an ionic, a non ionic and zwitterionic surfactant.

It is a further object of the present invention to disclose the pharmaceutical composition, wherein the protective agent is selected from a group consisting of antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones, biochemical intermediates, and any combination thereof.

It is a further object of the present invention to disclose the pharmaceutical composition, wherein the dispersing agent is selected from a group consisting of a surfactant, a cholesteryl ester, a fatty acid, a phospholipid, a carbohydrate, a protein and a mixture thereof.

It is a further object of the present invention to disclose the pharmaceutical composition, wherein the surfactant is selected from a group consisting of a natural ionic surfactant, a synthetic ionic surfactant, a natural non-ionic surfactant, a synthetic non-ionic surfactant and a mixture thereof.

It is a further object of the present invention to disclose the pharmaceutical composition, wherein the composition has disintegration time of between about 3 minutes and about 60 minutes in the vagina.

It is a further object of the present invention to disclose the pharmaceutical composition, wherein the composition has a disintegration time of less than about 3 minutes in water at room temperature.

It is a further object of the present invention to disclose the pharmaceutical composition, wherein the composition has disintegration time of between about 3 minutes and about 15 minutes in water at room temperature.

It is a further object of the present invention to disclose the pharmaceutical composition, wherein the composition is configured for administration in a dosage unit of between about 10 mg to about 200 mg of danazol.

It is a further object of the present invention to disclose the pharmaceutical composition, wherein the composition is configured for administration in a dosage unit of less than about 10 mg of danazol.

It is a further object of the present invention to disclose the pharmaceutical composition, wherein the at least one NSAID is selected from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt or derivative thereof and any combination thereof.

It is a further object of the present invention to disclose the pharmaceutical composition, wherein the at least one NSAID is further selected from a group consisting of: aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, and a pharmaceutically acceptable salt or derivative thereof and any combination thereof.

It is a further object of the present invention to disclose the pharmaceutical composition, wherein the at least one NSAID is configured for administration in dosage unit of between about 50 mg to about 300 mg.

It is a further object of the present invention to disclose the pharmaceutical composition, wherein the at least one NSAID is configured for administration in a daily dosage of between about 5 mg to about 2000 mg.

It is a further object of the present invention to disclose the pharmaceutical composition, wherein the composition is configured for administration vaginally one to six times per day.

It is a further object of the present invention to disclose the pharmaceutical composition, wherein the Diclofenac is configured for administration in a dosage unit of between about 10 mg to about 50 mg, two times per day, or in a daily dosage unit of between about 10 mg to about 100 mg.

It is a further object of the present invention to disclose the pharmaceutical composition, wherein the Naproxen is configured for administration in a dosage unit of between about 25 mg to about 500 mg, one to two times per day.

It is a further object of the present invention to disclose the pharmaceutical composition, wherein the Piroxicam is configured for administration in a daily dosage unit of between about 5 mg to about 20 mg.

It is a further object of the present invention to disclose the pharmaceutical composition, wherein the Ibuprofen is configured for administration in a dosage unit of between about 20 mg to about 200 mg, one to three times per day.

It is a further object of the present invention to disclose the pharmaceutical composition, wherein the Indomethacin is configured for administration in a dosage unit of between about 5 mg to about 25 mg, one to six times per day.

It is a further object of the present invention to disclose the pharmaceutical composition, wherein the Mefenamic acid is configured for administration in a dosage unit of between about 25 mg to about 500 mg, one to four times per day.

It is a further object of the present invention to disclose the pharmaceutical composition, wherein the composition has a rapid disintegration time in the vagina.

It is a further object of the present invention to disclose the pharmaceutical composition, wherein the at least one NSAID has a lower absolute or systemic bioavailability ratio in the serum or blood stream as compared to conventional oral NSAID compositions comprising comparable amounts of the at least one NSAID.

It is a further object of the present invention to disclose the pharmaceutical composition, wherein the danazol has a lower absolute or systemic bioavailability ratio in the serum or blood stream as compared to conventional oral danazol compositions comprising comparable amounts of the danazol ingredient.

It is a further object of the present invention to disclose the pharmaceutical composition, wherein the at least one NSAID has a higher relative bioavailability ratio at the endometrial and/or vaginal tissue as compared to conventional oral NSAID compositions comprising comparable amounts of the at least one NSAID.

It is a further object of the present invention to disclose the pharmaceutical composition, wherein the danazol has a higher relative bioavailability ratio at the endometrial and/or vaginal tissue as compared to conventional oral danazol compositions comprising comparable amounts of danazol.

It is a further object of the present invention to disclose the pharmaceutical composition, wherein the composition is characterized by a property selected from a group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof.

It is a further object of the present invention to disclose the pharmaceutical composition, wherein the composition has an improved or enhanced property selected from the group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof relative to conventional oral compositions comprising comparable amounts of danazol or the at least one NSAID.

It is a further object of the present invention to disclose the pharmaceutical composition, wherein the composition comprises micronized danazol.

It is a further object of the present invention to disclose the pharmaceutical composition, wherein the composition confers at least one synergistic effect with respect to treatment of a gynecological condition selected from a group consisting of endometriosis, adenomyosis, menorrhagia, dysmenorrheal, menstruation pain and any combination thereof by having more than an additive effect.

It is a further object of the present invention to disclose the pharmaceutical composition, wherein the composition confers a synergistic effect with respect to the efficacy of the composition in treatment of a gynecological condition selected from a group consisting of endometriosis, adenomyosis, menorrhagia, dysmenorrheal, menstruation pains and any combination thereof, by having a more than additive effect relative to the effect conferred when comparable amounts of the danazol, or the at least one NSAID is administered separately.

It is a further object of the present invention to disclose the pharmaceutical composition, wherein the “add-on” formulation is configured as a tampon coated with a film comprising the composition of claim 1 or any of its dependent claims.

It is a further object of the present invention to disclose the pharmaceutical composition, wherein the composition is additionally provided with an applicator or a dispensing device suitable for vaginal administration.

It is a further object of the present invention to disclose the pharmaceutical composition, wherein the applicator is a conventional applicator suitable for vaginal administration of a solid dosage form.

It is a further object of the present invention to disclose the pharmaceutical composition, wherein the dispensing device is suitable for vaginal administration of a foam or mousse form.

It is a further object of the present invention to disclose the pharmaceutical composition, wherein the dispensing device is selected from an aerosol and a non-aerosol dispensing device.

It is a further object of the present invention to disclose the pharmaceutical composition, wherein the dispensing device is an aerosol dispensing device, further comprising an aerosol propellant.

It is a further object of the present invention to disclose a pharmaceutical composition useful for treating a gynecological condition comprising (a) an active ingredient selected from a group consisting of at least one non-steroidal anti-inflammatory drug (NSAID), danazol and a combination thereof, (b) at least one pharmaceutically acceptable carrier or excipient, wherein the composition is configured as an “add-on” to a substrate formulation adapted to be vaginally administrable, further wherein the composition is in an immediate release form.

It is a further object of the present invention to disclose the pharmaceutical composition, wherein the composition is formulated as a film or layer at least partially coating the substrate.

It is a further object of the present invention to disclose the pharmaceutical composition, wherein the composition is adapted to disintegrate from the substrate upon contact with the vaginal epithelium, menstrual fluid and/or vaginal discharges.

It is a further object of the present invention to disclose the pharmaceutical composition, wherein the substrate is selected from a group consisting of a tampon, a catamenial tampon, a pessary, an absorbent article, a patch and a pad.

It is a further object of the present invention to disclose the pharmaceutical composition, wherein the add-on formulation is configured to encompass both activities of absorbing menstrual fluid and, at the same time, releasing effective amounts of at least one ingredient selected from a group consisting of: danazol, and at least one NSAID compound, directly to the vaginal tissue.

It is a further object of the present invention to disclose the pharmaceutical composition, wherein the composition is further configured to incorporate on and/or into the tampon materials in a form selected from a group consisting of: fugitive, loosely adhered, bound, and any combination thereof.

It is a further object of the present invention to disclose the pharmaceutical composition, wherein the carrier or excipient is selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

It is a further object of the present invention to disclose a kit useful for treating gynecological disorders, especially at least one of endometriosis, adenomyosis, mennorhagia, dysmenorrhea and menstruation pains, wherein the kit comprising: (a) a plurality of solid dosage forms, each solid dosage form containing (i) effective amount of at least one active ingredient selected from a group consisting of danazol, at least one non steroidal anti inflammatory drug (NSAID) and a combination thereof, (ii) at least one pharmaceutically acceptable carrier or excipient; (b) an applicator suitable for vaginal administration; and, (c) instructions for use of the composition and the applicator; wherein the composition is adapted to be vaginally administrable further wherein the composition is in an immediate release form.

It is a further object of the present invention to disclose the kit, wherein the composition is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

It is a further object of the present invention to disclose the kit, wherein the carrier or excipient is selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

It is a further object of the present invention to disclose the kit, wherein the at least one NSAID is selected from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt or derivative thereof and any combination thereof.

It is a further object of the present invention to disclose the kit, wherein the at least one NSAID is further selected from a group consisting of: aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, and a pharmaceutically acceptable salt or derivative thereof and any combination thereof.

It is a further object of the present invention to disclose a pharmaceutical composition for vaginal administration, prepared by steps of: (a) preparing a mixture comprising (i) an effective amount of at least one active ingredient selected from a group consisting of danazol, at least one NSAID and a combination thereof, and (ii) at least one pharmaceutically acceptable carrier or excipient and, (b) forming the composition in an immediate release form.

It is a further object of the present invention to disclose a composition for vaginal administration prepared by the steps as defined above, wherein the mixture is further prepared by steps of forming the composition in a form selected from a group consisting of a solid dosage form, foam and mousse type delivery form.

It is a further object of the present invention to disclose a composition for vaginal administration prepared by the steps as defined above, wherein the mixture is further prepared by steps of forming the composition in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

It is a further object of the present invention to disclose a composition for vaginal administration prepared by the steps as defined above, wherein the mixture is further prepared by steps of: (a) adding water to the mixture to prepare a second mixture; (b) granulating the second mixture; (c) drying the granulation mixture; and, (d) compressing the granules so as to form carriers.

It is a further object of the present invention to disclose a composition for vaginal administration prepared by the steps as defined above, wherein the mixture is further prepared by steps of: (a) preparing a first mixture containing a polymeric solution comprising at least one polymer and at least one solvent; (b) preparing a second mixture by adding the danazol or the at least one NSAID or a combination thereof to the first mixture; and (c) preparing a thin film type form or mucoadhesive type form by casting the second mixture into predetermined containers and allowing it to dry.

It is a further object of the present invention to disclose a composition for vaginal administration prepared by the steps as defined above, wherein the composition is further prepared by steps of adding at least one of Carbopol solution and/or glycerine into the first mixture.

It is a further object of the present invention to disclose a composition for vaginal administration prepared by the steps as defined above, wherein the composition is further prepared by steps of cutting the dried film into predetermined units.

It is a further object of the present invention to disclose a composition for vaginal administration prepared by the steps as defined above, wherein the mixture is further prepared by steps of a wet granulation process.

It is a further object of the present invention to disclose a composition for vaginal administration prepared by the steps as defined above, wherein the second mixture additionally comprises a lubricating agent, such as Mg stearate.

It is a further object of the present invention to disclose a composition for vaginal administration prepared by the steps as defined above, wherein the composition is further prepared by steps of incorporating into the mixture at least one carrier or excipient selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents, add-on” formulation excipients and any combination thereof.

It is a further object of the present invention to disclose a composition for vaginal administration prepared by the steps as defined above, wherein the composition is further prepared by steps of preparing the mixture with between about 10 mg to about 250 mg of danazol.

It is a further object of the present invention to disclose a composition for vaginal administration prepared by the steps as defined above, wherein the composition is further prepared by steps of preparing the mixture with between about 50 mg to about 300 mg of the at least one NSAID ingredient.

It is a further object of the present invention to disclose a method for treating a gynecological condition, especially endometriosis, adenomyosis, dysmenorrhea, mennorhagia and menstruation pains, wherein the method comprises steps of: (a) formulating a composition comprising at least one active ingredient selected from a group consisting of at least one NSAID, danazol, and a combination thereof, and at least one carrier or excipient; (b) administering the composition vaginally at a therapeutically effective dosage; wherein the composing is formulated in an immediate release form.

It is a further object of the present invention to disclose the method, comprising an additional step of formulating the composition as a solid dosage form, foam or mousse type delivery form.

It is a further object of the present invention to disclose the method, comprising an additional step of formulating the composition in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

It is a further object of the present invention to disclose the method, comprising an additional step of selecting the carrier or excipient from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

It is a further object of the present invention to disclose the method, wherein the composition comprises between about 5 to about 12 wt. % effervescent excipient.

It is a further object of the present invention to disclose the method, comprising an additional step of selecting the at least one Thin Film type or PharmFilm type or mucoadhesive type excipient from a group consisting of: hydroxypropyl methyl cellulose (HPMC) K4M, Carbopol 934, hyaluronic acid, Gelatin, Mannitol, Citric acid, Chitosan, Sodium Alignate, Cyclodextrin and combination thereof.

It is a further object of the present invention to disclose the method, comprising an additional step of selecting the at least one FDT type excipient from a group consisting of: Mannitol, Lactose, Erythritol, Xylitol, Glucose, Sucrose, Sorbitol, Maltitol, Trehalose, Maltose and mixtures thereof.

It is a further object of the present invention to disclose the method, comprising an additional step of selecting the bioadhesive type agent from a group consisting of Carbopol 971, hyaluronic acid, HPMC, hydrophilic polymers such as polyox, PEG (Polyethylene glycol), hydrogels, cellulose derivatives such as, sodium alginate, carboxymethylcellulose, hydroxymethylcellulose, methylcellulose, hydroxypropyl methyl cellulose (HPMC) K4M, Carbopol 934, Gelatin, Mannitol, Citric acid, Chitosan, Sodium Alignate, Cyclodextrin and any mixture thereof.

It is a further object of the present invention to disclose the method, comprising an additional step of selecting the probiotic agent from a group consisting of lactic-acid bacteria such as Lactobacillus rhamnosus, bifidobacterium, streptococcus, factors that stimulate the growth of protective organisms, enzymes, vitamins and cellular factors and mixtures thereof.

It is a further object of the present invention to disclose the method, comprising an additional step of selecting the acidifying agent from a group consisting of boric acid, acetic acid, glacial acetic acid, fumaric acid, diluted hydrochloric acid, citric acid, lactic acid, malic acid, nitric acid, phosphoric acid, sulfuric acid, tartaric acid, acidic buffer and mild organic acids approved for pharmaceutical applications and any combination thereof.

It is a further object of the present invention to disclose the method, comprising an additional step of selecting the surfactant from a group consisting of: a hydrophilic, a non hydrophilic, a polymeric, an ionic, a non ionic and zwitterionic surfactant.

It is a further object of the present invention to disclose the method, comprising an additional step of selecting the protective agent from a group consisting of antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones, biochemical intermediates, and any combination thereof.

It is a further object of the present invention to disclose the method, comprising an additional step of selecting the dispersing agent from a group consisting of a surfactant, a cholesteryl ester, a fatty acid, a phospholipid, a carbohydrate, a protein and a mixture thereof.

It is a further object of the present invention to disclose the method, comprising an additional step of selecting the surfactant from a group consisting of a natural ionic surfactant, a synthetic ionic surfactant, a natural non-ionic surfactant, a synthetic non-ionic surfactant and a mixture thereof.

It is a further object of the present invention to disclose the method, comprising an additional step of reducing menstrual blood loss by at least about 10%.

It is a further object of the present invention to disclose the method, comprising an additional step of configuring the composition for administration in a dosage unit of between about 10 mg to about 200 mg of danazol.

It is a further object of the present invention to disclose the method, comprising an additional step of configuring the composition for administration in a dosage unit of less than about 10 mg of danazol.

It is a further object of the present invention to disclose the method, comprising an additional step of selecting the at least one NSAID from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt or derivative thereof and any combination thereof.

It is a further object of the present invention to disclose the method, comprising an additional step of selecting the at least one NSAID from a group consisting of: aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, and a pharmaceutically acceptable salt or derivative thereof and any combination thereof.

It is a further object of the present invention to disclose the method, comprising an additional step of configuring the composition for administration in a dosage unit of between about 50 mg to about 300 mg of the at least one NSAID.

It is a further object of the present invention to disclose the method, comprising an additional step of configuring the composition for administration in a daily dosage of between about 5 mg to about 2000 mg of the at least one NSAID.

It is a further object of the present invention to disclose the method, comprising an additional step of configuring the composition for administration vaginally one to six times per day.

It is a further object of the present invention to disclose the method, comprising an additional step of formulating the composition with a rapid disintegration time in the vagina.

It is a further object of the present invention to disclose the method, comprising an additional step of formulating the composition with a property selected from a group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof.

It is a further object of the present invention to disclose the method, comprising an additional step of formulating the composition with an improved or enhanced property selected from the group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof relative to conventional oral compositions comprising comparable amounts of danazol or the at least one NSAID.

It is a further object of the present invention to disclose the method, comprising an additional step of formulating the composition with micronized danazol.

It is a further object of the present invention to disclose the method, comprising an additional step of conferring at least one synergistic effect with respect to treatment of a gynecological condition selected from a group consisting of endometriosis, adenomyosis, menorrhagia, dysmenorrheal, menstruation pain and any combination thereof by having more than an additive effect.

It is a further object of the present invention to disclose the method, comprising an additional step of conferring a synergistic effect with respect to the efficacy of the composition in treatment of a gynecological condition selected from a group consisting of endometriosis, adenomyosis, menorrhagia, dysmenorrheal, menstruation pains and any combination thereof, by having a more than additive effect relative to the effect conferred when comparable amounts of the danazol, or the at least one NSAID is administered separately.

It is a further object of the present invention to disclose the method, comprising an additional step of configuring the “add-on” formulation as a tampon coated with a film comprising the composition of claim 1 or any of its dependent claims.

It is a further object of the present invention to disclose the method, comprising an additional step of providing the composition with an applicator or a dispensing device suitable for vaginal administration.

It is a further object of the present invention to disclose the method, wherein the applicator is a conventional applicator suitable for vaginal administration of a solid dosage form.

It is a further object of the present invention to disclose the method, wherein the dispensing device is suitable for vaginal administration of a foam or mousse form.

It is a further object of the present invention to disclose the method, comprising an additional step of selecting the dispensing device from a group consisting of an aerosol and a non-aerosol dispensing device.

It is a further object of the present invention to disclose the method, wherein the dispensing device is an aerosol dispensing device, further comprising an aerosol propellant.

It is a further object of the present invention to disclose a use of a vaginally administrable composition, for the manufacture of a medicament for the treatment of a gynecological disorder, especially endometriosis, adenomyosis, menorrhagia, dysmenorrhea and menstruation pains, the composition comprising (a) an effective amount of at least one active ingredient selected form a group consisting of at least one non-steroidal anti-inflammatory drug (NSAID), danazol and a combination thereof, (b) at least one pharmaceutically acceptable carrier or excipient, wherein the composition is in an immediate release form.

It is a further object of the present invention to disclose the use, wherein the composition is adapted to reduce menstrual blood loss by at least about 10%.

It is a further object of the present invention to disclose the use, wherein the composition is formulated in a form selected from a group consisting of a solid dosage form, foam and a mousse.

It is a further object of the present invention to disclose the use, wherein the composition is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

It is a further object of the present invention to disclose the use, wherein the carrier or excipient is selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

It is a further object of the present invention to disclose the use, wherein the composition comprises between about 5 to about 12 wt. % effervescent excipient.

It is a further object of the present invention to disclose the use, wherein the at least one Thin Film type or PharmFilm type or mucoadhesive type excipient is selected from a group consisting of: hydroxypropyl methyl cellulose (HPMC) K4M, Carbopol 934, hyaluronic acid, Gelatin, Mannitol, Citric acid, Chitosan, Sodium Alignate, Cyclodextrin and combination thereof.

It is a further object of the present invention to disclose the use, wherein the at least one FDT type excipient is selected from a group consisting of: Mannitol, Lactose, Erythritol, Xylitol, Glucose, Sucrose, Sorbitol, Maltitol, Trehalose, Maltose and mixtures thereof.

It is a further object of the present invention to disclose the use, wherein the bioadhesive type agent is selected from a group consisting of Carbopol 971, hyaluronic acid, HPMC, hydrophilic polymers such as polyox, PEG (Polyethylene glycol), hydrogels, cellulose derivatives such as, sodium alginate, carboxymethylcellulose, hydroxymethylcellulose, methylcellulose, hydroxypropyl methyl cellulose (HPMC) K4M, Carbopol 934, Gelatin, Mannitol, Citric acid, Chitosan, Sodium Alignate, Cyclodextrin and any mixture thereof.

It is a further object of the present invention to disclose the use, wherein the probiotic agent is selected from a group consisting of lactic-acid bacteria such as Lactobacillus rhamnosus, bifidobacterium, streptococcus, factors that stimulate the growth of protective organisms, enzymes, vitamins and cellular factors and mixtures thereof.

It is a further object of the present invention to disclose the use, wherein the acidifying agent is selected from a group consisting of boric acid, acetic acid, glacial acetic acid, fumaric acid, diluted hydrochloric acid, citric acid, lactic acid, malic acid, nitric acid, phosphoric acid, sulfuric acid, tartaric acid, acidic buffer and mild organic acids approved for pharmaceutical applications and any combination thereof.

It is a further object of the present invention to disclose the use, wherein the surfactant is selected from a group consisting of: a hydrophilic, a non hydrophilic, a polymeric, an ionic, a non ionic and zwitterionic surfactant.

It is a further object of the present invention to disclose the use, wherein the protective agent is selected from a group consisting of antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones, biochemical intermediates, and any combination thereof.

It is a further object of the present invention to disclose the use, wherein the dispersing agent is selected from a group consisting of a surfactant, a cholesteryl ester, a fatty acid, a phospholipid, a carbohydrate, a protein and a mixture thereof.

It is a further object of the present invention to disclose the use, wherein the composition is characterized by a property selected from a group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof.

It is a further object of the present invention to disclose the use, wherein the composition confers at least one synergistic effect with respect to treatment of a gynecological condition selected from a group consisting of endometriosis, adenomyosis, menorrhagia, dysmenorrheal, menstruation pain and any combination thereof by having more than an additive effect.

It is a further object of the present invention to disclose the use, wherein the composition confers a synergistic effect with respect to the efficacy of the composition in treatment of a gynecological condition selected from a group consisting of endometriosis, adenomyosis, menorrhagia, dysmenorrheal, menstruation pains and any combination thereof, by having a more than additive effect relative to the effect conferred when comparable amounts of the danazol, or the at least one NSAID is administered separately.

It is a further object of the present invention to disclose the use, wherein the composition is formulated as an “add-on” formulation comprising a tampon coated with a film or layer comprising the composition of claim 1 or any of its dependent claims.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide compositions and methods of treating endometriosis, adenomyosis, mennorhagia, dysmenorrhea and menstruation pains.

The present invention provides a composition useful for treating gynecological disorders, especially endometriosis, adenomyosis, mennorhagia, dysmenorrhea and menstruation pains. The composition comprises an active ingredient selected from a group consisting of at least one non steroidal anti inflammatory drug (NSAID), danasol and a combination thereof and at least one pharmaceutically acceptable carrier or excipient. It is a core aspect of the invention to provide the aforementioned composition adapted to be vaginally administrable. In a further core aspect the composition is in an immediate release form.

Thus, an immediate release NSAID or NSAID combined with danazol composition for vaginal application is disclosed. The vaginal composition of the present invention is preferably formed as a tablet which has fast dissolving properties in the vaginal environment. The fast dissolving properties may be attributed by formulating the tablet with an effervescent ingredient such as sodium bicarbonate or in an alternative embodiment, formulating the vaginal composition in a Fast Dissolving Tablet (FDT) type form. The immediate release vaginal formulations of the present invention unexpectedly use the advantage of enhanced disintegration time in the vagina during the bleeding period, and especially in a heavy bleeding period. The menstruation period commonly have significant disadvantages for most drugs directed to the vaginal area, and especially to the majority of which, that are characterized by slow release properties. The composition of the present invention is formulated to be most efficacious at the vaginal tissue environment including during menstruation period to treat endometriosis, adenomyosis, mennorhagia, dysmenorrhea and menstruation pains symptoms.

According to certain embodiments the vaginal pharmaceutical composition is characterized by having at least one of the properties selected from a group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof. These properties contribute to the suitability and compliance of the composition to improve gynecological condition symptoms, particularly endometriosis, adenomyosis, mennorhagia, dysmenorrhea and menstruation pains symptoms.

In a further embodiment, the disintegrated tablet residuals have a bioadhesive property so as to adhere to the vaginal tissue.

According to a main embodiment of the present invention, the composition is formulated in an immediate release form and has a rapid disintegration time in the vagina.

Vaginal administration has several advantages. The vaginal area is characterized by a rich blood supply, resulting in rapid and steady uptake of drugs, lower serum concentrations than oral drug delivery and a decrease in first-pass liver metabolism, which allows low doses with fewer side effects.

By using vaginal administration, the same effect is obtained with much lower serum concentrations of the therapeutic agent. A higher ovarian and uterine concentrations of the active ingredient is achieved after vaginal than after oral administration. Furthermore, with the vaginal route, the same effect is obtained without inhibition of ovulation and other general side effects often observed with the oral danazol therapy.

It is herein further acknowledged that transmucosal routes of drug delivery (i.e., the mucosal linings of the vaginal or rectal cavity) offer distinct advantages over oral administration for systemic effects. These advantages include possible bypass of first pass effect, avoidance of presystemic elimination within the gastrointestinal tract, and, depending on the particular drug, a better enzymatic flora for drug absorption.

In other embodiment of the invention, in order to prevent the progressive suffer, disabling dysmenorrhea and pelvic pain at the time of menses, administration of a danazol is combined with other medical or surgical treatments for endometriosis as non-steroidal anti-inflammatory compound (NSAID).

In accordance with a further embodiment of the invention, treatment of menorrhagia with prostaglandin synthetase inhibitor such as NSAID, results in a significant reduction in menstrual blood loss in women with primary menorrhagia as well as with Intrauterine device (IUD)-induced excessive menstrual blood loss, as compared to the reduction obtained by treatment with antifibrinolytic agents or oral contraceptives.

It is also within the scope of the present invention to provide a vaginally administrable composition useful for treating gynecological disorders, especially endometriosis, adenomyosis, mennorhagia, dysmenorrhea and menstruation pains, comprising at least one NSAID compound, formulated in a rapid release form.

In a further embodiment, the therapeutically active agent is an anti-inflammatory agent. Such an agent may be, for example, any non-steroidal anti-inflammatory agent (NSAID), antipyrin and pharmaceutically acceptable derivatives thereof.

Nonsteroidal anti-inflammatory drugs, usually abbreviated to NSAIDs or NAIDs, but also referred to as nonsteroidal anti-inflammatory agents/analgesics (NSAIAs) or nonsteroidal Anti-inflammatory medicines (NSAIMs), are drugs with analgesic and antipyretic effects and in higher doses have anti-inflammatory effects.

The term “nonsteroidal” is used to distinguish NSAIDs from steroids, which, among a broad range of other effects, have a similar eicosanoid-depressing, anti-inflammatory action. As analgesics, NSAIDs are unusual in that they are non-narcotic.

NSAIDs are usually indicated for the treatment of acute or chronic conditions where pain and inflammation are present.

According to one embodiment of the invention, the at least one NSAID is selected from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt or derivative thereof and any combination thereof.

Examples of the non-steroidal anti-inflammatory drug (NSAID) which is advantageously administered by the formulations of this invention include salicylic acid derivatives, such as, for example, aspirin; heteroaryl acetic acids, such as, for example, tolmetin, diclofenac, ketorolac; arylpropionic acids, such as, for example, ibuprofen, naproxen, flurbiprofen, ketoprofen, fenoprofen, oxaprozin; anthranilic acids (fenamates), such as, for example, mefenamic acid, meclofenamic acid, flufenamic acid; enolic acids, such as, for example, oxicams (e.g., piroxicam, tenoxicam), pyrazolidinediones (e.g., phenylbutazone, oxyphenthatrazone); alkanones, such as, for example, nabumetone. Among these, especially preferred, based on the current level of knowledge in the pharmacological arts, are ibuprofen, diclofenac, ketorolac, naproxen, flurbiprofen, ketoprofen and piroxicam. More generally, however, any of the government approved NSAIDs, such as listed in, for example, the most current edition of The Merck Index, may be advantageously used.

Examples of NSAIDs more preferably used in the present invention include aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, a pharmaceutically acceptable salt or derivative thereof and any combination thereof.

It is also within the scope of the present invention, that the vaginal composition of the present invention contains more than one NSAID compound. The use of a combination of at least two different NSAID molecules (such as mefenamic acid & naproxen or COX-inhibitor & ibuprofen) in the vaginal composition may reduce the dosage of each of the administered molecules or compounds, reduce side effects and have better efficacy of the composition as each molecule act on different step in the inflammatory cascade.

In is also with the scope of the present invention that, the pharmaceutical composition for vaginal administration is formulated in a form selected from a group consisting of a solid dosage form, foam and a mousse.

According to one embodiment, the present invention further provides a vaginal composition useful for treating gynecological disorders, especially mennorhagia, dysmenorrhea and menstruation pains, wherein the composition is in a solid dosage form comprising at least one non steroidal anti inflammatory drug (NSAID), danazol and a combination.

The present invention further provides a composition for vaginal administration with improved bioavailability, bioadhesiveness, disinteresting and solubility properties and rapid release and uptake of the drugs in the affected vaginal tissue to effectively treat disorders of the reproductive organs.

According to one aspect of the invention, the composition for vaginal administration comprising danazol, at least one NSAID, or a combination thereof, is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

In a certain embodiment, solid formulations can be configured as a coat layer upon a substrate such as a tampon adapted for vaginal administration. These configurations are herein referred to as “add-on” formulations.

According to another certain embodiment, The present invention provides fast dissolving tablet (FDT) formulation containing danazol, NSAID and a combination thereof.

It is herein acknowledged that FDT is defined by the food and drug administration (FDA) as a solid dosage form containing medical substances which disintegrates rapidly within a matter of seconds, when placed upon the tongue.

The present invention uses compositions containing danazol and/or at least one NSAID for vaginal application formulated in a FDT technology. As used herein, the FDT technology is also referred to as fast disintegrating tablet, fast dispersing tablet, rapid dissolving tablet, rapid melt tablet, quick disintegrating tablet and vaginally disintegrating tablet.

The aforesaid vaginal compositions formulated in a FDT form, comprising danazol, NSAID or a combination thereof has advantages over oral solutions by i.e. having better stability characteristics, more accurate dosing and lower volume and weight of the composition.

According to certain embodiments of the invention, such FDT formulations are prepared by several different methods including crystalline transition, phase transition, sublimation, spray drying, and direct compression.

According to a further embodiment of the present invention, the composition of the present invention formulated in a FDT form is made of highly hydrophilic materials and possesses highly porous structures for fast water absorption into the matrix. Such structures produce rapid disintegrating tablets which are highly advantageous for the compositions of the present invention. Pharmaceutical grade saccharides (or sugars) such as mannitol, sucrose, lactose, glucose, and xylitol are used in the present invention in making FDTs. More particularly, low compressibility saccharides such as mannitol, lactose, erythritol, xylitol and glucose exhibit rapid disintegration in the mucosal area of the vaginal cavity, and highly compressable saccharides such as sucrose, sorbitol, manitol trehalose and maltose yield high mechanical strength.

In certain aspects of the invention, the vaginal composition is formulated by a combination of a FDT type form with at least one bioadhesive excipient to enable rapid disintegration in the vaginal mucosal tissue on one hand, and on the other hand adhesiveness of the disintegrated composition residuals to the vaginal tissue, i.e. during menstruation to effectively treat the gynecological condition.

In a further certain embodiment, the vaginal pharmaceutical composition is configured in a Thin film type form.

Reference is now made to the Thin film type drug technology, often referred to as PharmFilm. It is herein acknowledged that the Thin film type drug technology uses a dissolving film or drug strip to administer drugs via absorption. In some embodiments of the invention, a film is prepared using hydrophilic polymers that rapidly dissolves on the vaginal cavity, delivering the drug via dissolution upon contact with the mucosal environment.

It should be emphasized that, up until now, thin film drug technology was used to administer drugs via absorption in the mouth (buccally or sublingually) and/or via the small intestines (enterically). In these orally administered drugs the film is rapidly dissolved on the tongue or buccal cavity, delivering the drug to the systemic circulation via dissolution when contact with liquid is made. These thin film strips are typically designed for oral administration, with the user placing the strip on or under the tongue (sublingual) or along the inside of the cheek (buccal). As the strip dissolves, the drug can enter the blood stream enterically, buccally or sublingually.

The present invention uses a dissolving film or drug strip to administer drugs (i.e. NSAID and/or danazol via absorption through the vaginal area. These thin film dosage forms may also herein refer to as buccal-type or mucoadhesive-type formulations or compositions.

In certain embodiments of the invention, the design of a thin film as a vaginal drug delivery offers several advantages over other modes of drug delivery, such as insertable tablets, softgels, liquids and ointment.

According to one aspect, the thin film drug delivery for vaginal application may improve the onset of action, lower the dosing, and enhance the efficacy and safety profile of the medicament, i.e. danazol and/or at least one NSAID compound. In a further embodiment of the invention, the vaginal composition in a thin film delivery form is more stable, durable and quicker dissolving than other conventional dosage forms.

In other certain embodiments of the invention, the use of a thin film technology for vaginal application not only may ensure more accurate administration of drugs but also can improve compliance of the dosage form due to it's inherent ease of administration.

Thin film type drug delivery is highly advantageous especially for drugs having poor solubility and/or high first pass metabolism such as danazol and NSAIDs. By using the vaginal route for the herein disclosed compositions in a thin film form, an improved bioavailability of the active ingredients, namely danazol and NSAID, is obtained.

The danazol and/or NSAID thin films provided by the present invention may deliver a convenient, quick-dissolving therapeutic dose contained within a film matrix that rapidly absorbs through the vaginal mucosa to ensure compliance.

In other words, it is herein acknowledged that solid dosage units for vaginal insertion are of the thin film drug delivery types and variations thereof, which use a dissolving film or drug strip to administer drugs via absorption. Some embodiments of the present invention exploit thin film or Pharmafilm technology currently used in the mouth (buccally or sublingually) and/or via the small intestines (enterically). The vaginal thin film drug delivery medium is a film prepared using hydrophilic polymers that rapidly dissolves on the mucosal environment of the vagina; delivering the drug to the systemic circulation via dissolution when contact with liquid or moisture is made.

Thus, as used herein, the term ‘thin film’ refers to a dissolving pharmaceutical dosage form, preferably a film or strip, used to administer selected drugs via absorption in a mucosal environment. Such a technology is also defined in the present invention as buccal type or mucoaddhesive or PharmFilm type delivery technology or platform.

Thin film type or mucoadhesive type excipients refer to compounds useful for thin film delivery including, but not limited to polysaccharides, polymers and other biochemical molecules adapted to have dual advantages including increasing the solubility and bioavailability of the drug, combined with avoidance of first pass metabolism. Examples of buccal type or thin film or mucoadhesive formulations may encompass Chitosan, Sodium alignate, Cyclodextrin, Hdroxypropyl methyl cellulose (HPMC) K4M, Carbopol 934, gelatin, mannitol, citric acid and mixtures thereof.

According to a further embodiment of the present invention, a cyclodextrin complex formulation containing danazol, at least one NSAID or a combination thereof is provided. Such a formulation is specifically suitable for increasing solubility of poorly dissolving drugs such as danazol and avoidance of first pass metabolism.

Other examples for thin film formulations which improve bioavailability and aqueous solubility include chitosan, sodium alginate and mixtures thereof, Diclofenac Sodium, chlorpheniramine maleate, metoprolol tartrate and propranolol hydrochloride.

According to a further embodiment of the present invention the vaginal composition has a lower systemic bioavailability ratio as compared to conventional oral and/or conventional vaginal danazol compositions comprising comparable amounts of danazol.

According to a further embodiment of the present invention the composition has a lower systemic bioavailability ratio as compared to conventional oral compositions comprising comparable amounts of said at least one NSAID.

According to a further embodiment of the present invention the composition has a lower absolute and/or systemic bioavailability ratio in the bloodstream or serum as compared to conventional oral and/or conventional vaginal danazol compositions comprising comparable amounts of danazol.

According to a further embodiment of the present invention the composition has a lower absolute and/or systemic bioavailability ratio in the bloodstream or serum as compared to conventional oral compositions comprising comparable amounts of said at least one NSAID.

According to a further embodiment of the present invention the composition has a higher relative bioavailability ratio at the endometrial and/or vaginal tissue as compared to conventional oral and/or conventional vaginal danazol compositions comprising comparable amounts of danazol.

According to a further embodiment of the present invention the composition has a higher relative bioavailability ratio at the endometrial and/or vaginal tissue as compared to conventional oral compositions comprising comparable amounts of said at least one NSAID.

According to a further embodiment of the present invention the composition has a lower absolute or systemic bioavailability ratio in the serum or blood stream as compared to conventional oral danazol or NSAID compositions comprising comparable amounts of danazol or NSAID.

According to a further embodiment of the present invention the composition has a higher relative bioavailability ratio at the endometrial and/or vaginal tissue as compared to conventional oral danazol or NSAID compositions comprising comparable amounts of danazol or NSAID.

According to a further embodiment of the present invention the serum concentration of danazol is 20 times lower than said serum danazol concentration after oral administration of about four times higher dosage.

According to a further embodiment of the present invention the endometrial and/or vaginal danazol concentration is about four times higher as compared to said danazol concentration after oral administration of comparable dosage of danazol.

According to a further embodiment of the present invention the danazol concentration in the bloodstream is undetectable.

According to a further embodiment of the present invention the NSAID concentration in the bloodstream is undetectable.

In a further main embodiment of the invention, the composition of danazol and at least one NSAID, vaginally administered, achieves a reduction in heavy menstrual bleeding in women of reproductive years.

In a further embodiment, the composition of the present invention is adapted to reduce menstruation pains or dysmenorrheal.

According to one embodiment of the present invention, the composition as defined above is adapted to reduce menstrual blood loss by about 10% to about 30%.

According to another embodiment of the present invention, the composition as defined above is adapted to reduce menstrual blood loss by at least about 30%.

According to a further embodiment of the present invention, the pharmaceutical composition is configured for administration in a daily dosage of between about 5 mg to about 2000 mg of said at least one NSAID.

According to a specific embodiment of the present invention, the pharmaceutical composition is configured for administration in a dosage unit of between about 50 mg to about 300 mg of said at least one NSAID.

According to a further embodiment of the present invention, the pharmaceutical composition is configured for administration vaginally one to six times per day.

In accordance with certain aspects of the present invention, the aforementioned vaginal composition is combined with enhanced bioavailability properties including rapid disintegration time, bioadhesiveness to the vaginal surface and improved solubility and stability of the danazol and at least one NSAID active ingredients.

The additional therapeutic agent, according to the invention, for the combined therapy with danazol of the estrogen-dependent disorders, is a non-steroidal anti-inflammatory compound (NSAID).

As used herein the term “Non Steroidal Anti-Inflammatory Drug” or “NSAID” refers to a compound, selected from a group comprising acetyl salicylic acid, indometacin, sulindac, phenylbutazone, diclofenac, fentiazac, ketorolac, piroxicam, tenoxicam, mecoxicam, meloxicam, cinnoxicam, ibufenac, ibuprofen, naproxen, ketoprofen, nabumetone, niflumic acid and nimesulide, or a pharmaceutically acceptable salt thereof or mixtures thereof. Other preferred NSAIDs include piroxicam, tenoxicam, mecoxicam, meloxicam, ibufenac, ibuprofen, naproxen and ketoprofen, or a pharmaceutically acceptable salt thereof, or mixtures thereof.

Useful NSAIDs can be selected from suitable acidic and nonacidic compounds. Suitable acidic compounds include carboxylic acids and enolic acids. Suitable nonacidic compounds include, for example, nabumetone, tiaramide, proquazone, bufexamac, flumizole, epirazole, tinoridine, timegadine and dapsone.

According to certain aspects of the invention, suitable carboxylic acid NSAIDs may include, salicylic acids and esters thereof, such as aspirin, diflunisal, benorylate and fosfosal; acetic acids, including phenylacetic acids such as diclofenac, alclofenac and fenclofenac, and carbo- and heterocyclic acetic acids such as etodolac, indomethacin, sulindac, rolmerin, fentiazac and tilomisole; propionic acids, such as carprofen, fenbufen, flurbiprofen, ketoprofen, oxaprozin, suprofen, tiaprofenic acid, ibuprofen, naproxen, fenoprofen, indoprofen, pirprofen; and fenamic acids, such as flufenamic, mefenamic, meclofenamic and niflumic.

According to other embodiments of the invention, suitable enolic acid NSAIDs include, for example, pyrazolones such as oxyphenbutazone, phenylbutazone, apazone and feprazone, and oxicams such as piroxicam, sudoxicam, isoxicam and tenoxicam.

An effective amount of a NSAID, according to one embodiment of the present invention is generally the one commonly used in oral therapy for such compound as the maximal amount and about 10% of the aforementioned amount as the minimal dose. Proposed NSAID doses that may be used in the present invention are detailed herein after, for instance, an effective amount of naproxen may be in the range of about 25 mg to about 500 mg once or twice per day. An effective amount of piroxicam may be in the range of about 5 mg to about 20 mg once per day. An effective amount of acetyl salicylic acid may be in the range of about 150 to about 1000 mg once or twice per day. An effective amount of diclofena may be in the range of about 10 mg to about 50 mg twice per day or in the range of about 10 mg to about 100 mg once per day. An effective amount of ibuprofen may be in the range of about 20 mg to about 200 mg once or twice or three times per day. An effective amount of Indometacin may be in the range of about 5 mg to about 25 mg once or twice or three or four or five or six times per day. An effective amount of mefenamic acid may be in the range of about 25 mg to about 500 mg once or twice or three or four times per day.

In accordance with main aspects of the invention, the combination of the aforementioned improved bioavailability properties of the composition comprising at least one NSAID and danazol provide synergistic effects with respect to the formulation efficacy and effectiveness in treatment of gynecological ailments such as endometriosis, adenomyosis, dysmenorrheal, menorrhagia and menstruation pains by having a more than additive effect.

More specifically, the composition of the present invention, comprising the combination of at least one NSAID and danazol, confers a synergistic effect with respect to the efficacy of said composition in improving endometriosis, adenomyosis, menorrhagia, dysmenorrhea and menstruation pains symptoms by having a more than additive effect relative to the effect conferred when comparable amounts of said danazol and said at least one NSAID are administered separately.

The aforementioned synergistic effect may be attributed to the fact that NSAIDs and danazol possess different pharmacological mechanisms for decreasing menstruation bleeding. Furthermore, danazol has therapeutic effects on the bleeding symptoms, while NSAIDs are more effective in treating the pain symptoms, thus the simultaneous administration of both active compounds incorporated into one composition directly to the target organ has improved efficacy with respect to endometriosis, adenomyosis, menorrhagia, dysmenorrhea and menstruation pains, which is more than additive.

According to a further embodiment, the composition of the present invention has improved drug release ratio and/or increased mucoadhesive strength relative to conventional oral compositions comprising comparable amounts of danazol or NSAID.

In accordance with certain preferred embodiments of the invention, the vaginally administered danazol combined with NSAID carrier has a rapid disintegration time in the vagina. The rapid disintegration time of the carrier may be attributed to the immediate release properties of this composition.

In a main embodiment of the invention, the at least one NSAID or at least one NSAID combined with danazol composition comprises an effervescent ingredient. It is a core principle of the invention to provide the carrier composition especially suitable for the vaginal and uterine tissues since the carrier is formulated to dissolve immediately in the vaginal environment.

Thus, in certain main embodiments, the compositions of the present invention contain an effervescent component providing a short disintegration time, effective release and a rapid uptake of the active ingredients in the vaginal and uterine area. The aforementioned compositions are further adapted to the vaginal and uterine environment in that no tablet residues remain after each treatment period. The delivery of the drug, intra vaginally as herein disclosed, is directed close to the disease site and is thus less systemic than orally administered drugs or compositions. Since the method of delivery is relatively localized, lower doses may be given to the patient, whilst maintaining the therapeutic effect.

In accordance with a further embodiment of the present invention, the vaginally administered danazol, NSAID or a carrier comprising a combination of both ingredients, comprises between about 5% to about 12 wt. % effervescent excipient. Preferably, the aforementioned compositions for vaginal administration comprise between about 6 to about 8 wt. % effervescent excipient.

In a further aspect, the present invention provides a pharmaceutical composition for the treatment of a gynecological disorder in a form selected from foam and mousse comprising: a) a pharmaceutical agent selected from a group consisting of at least one NSAID, danazol and a combination thereof; b) a pharmaceutically acceptable carrier comprising at least one dispersing agent that is a foam forming agent.

In certain embodiments the dispersing agent that is a foam forming agent is selected from a surfactant, a cholesteryl ester, a fatty acid, a phospholipid, a carbohydrate a protein and a combination thereof.

In certain embodiments the pharmaceutical composition is provided as an aerosol foam or mousse and the composition further comprises a propellant. The propellant used may be chosen from conventional aerosol propellants. Thus, the propellant may be selected from propane, butane, dichloro difluoro methane, dichloro tetrafluoro ethane, octafluoro cyclobutane, and mixtures of two or more thereof. The propellant may be present in amounts of about 3 to about 30% w/w.

In other embodiments the composition is provided as non-aerosol foam or mousse.

According to a further embodiment of the present invention the vaginally administrable NSAID and/or NSAID combined with danazol composition, is used in the manufacture of a medicament for the treatment of menorrhagia and menstruation pains. Such a composition comprises an effective amount of NSAID and/or danazol, and has a rapid disintegration time in the vagina.

According to a further embodiment, the composition is administered in a daily unit dosage of about 50 mg to about 300 mg of NSAID.

According to a further embodiment, the composition is administered in a daily unit dosage of about 10 mg to about 50 mg of NSAID.

According to a further embodiment, the composition is administered in a daily unit dosage of about 25 mg to about 500 mg of NSAID.

According to a further embodiment, the composition is administered in a daily unit dosage of about 20 mg to about 200 mg of NSAID.

According to a further embodiment, the composition is administered in a daily unit dosage of about 5 mg to about 25 mg of NSAID.

In some embodiments of the present invention, NSAID doses of up to 200 mg per day are released over a short period of time for rapid and effective absorbance in the vaginal tissue.

According to a further embodiment of the present invention, the vaginally administrable composition comprises NSAID and at least one FDT excipient.

According to a further embodiment of the present invention, the vaginally administrable composition comprises between about 10 mg to about 200 mg of danazol. Preferably, the aforementioned composition comprises between about 50 mg to about 250 mg of danazol. Alternatively, the composition comprises less then about 10 mg of danazol.

According to a further embodiment, the composition is administered in a daily unit dosage of about 10 mg to about 200 mg of danazol.

In some embodiments of the present invention, danazol doses of up to 100 mg per day are released over a short period of time for rapid and effective absorbance in the vaginal tissue.

It is herein acknowledged that danazol is a steroidal drug with a very low aqueous solubility and poor bioavailability. To enhance the bioavailability of the danazol compound within the present composition, it is therefore advantageously provided in a form that promotes its rapid dissolution and efficient uptake in the intravaginal aqueous area.

Thus according to one embodiment of the present invention, the vaginally administrable composition comprises danazol and at least one mucoadhesive or a thin film delivery type excipient.

According to a further embodiment of the present invention, the vaginally administrable composition comprises danazol and at least one FDT ingredient.

As used herein the term “bioavailability” refers to a pharmacokinetic property which is used to describe the fraction of an administered dose of a drug that reaches the systemic circulation. It is herein acknowledged, that when a medication is administered intravenously, its bioavailability is defined as 100%. However, when a medication is administered via other routes (such as orally), its bioavailability decreases.

The term “absolute bioavailability” used herein refers to the bioavailability of an active drug in systemic circulation or bloodstream following non-intravenous administration (i.e., after oral, rectal, transdermal, subcutaneous, vaginal or sublingual administration), relative to the bioavailability of the same drug following intravenous administration. In pharmacology, the absolute bioavailability of a drug is determined by pharmacokinetic parameters defining the plasma drug concentration versus time plot for the drug after both intravenous (IV) and non-intravenous administration. The absolute bioavailability may be defined as the dose-corrected area under curve (AUC) obtained by non-intravenous divided by AUC obtained by intravenous administration. For example, the formula for calculating the bioavailability (F) for a drug administered by the oral route (po) is given below:

$$F = \frac{{[ AUC ]}_{po} * {dose}_{IV}}{{[ AUC ]}_{IV} * {dose}_{po}}$$

According to some embodiments of the present invention, the absolute bioavailability of the composition refers to the fraction of the drug absorbed through vaginal administration compared with the corresponding intravenous administration of the same drug. It is within the scope of the present invention that this ratio is compared to the ratio obtained by oral application of danazol and/or NSAID or to other slow release conventional vaginal compositions.

The term “relative bioavailability” used herein refers to the bioavailability (estimated as the AUC defined above) of a certain drug relative to the bioavailability of a different formulation of the same drug, for example the same drug formulated for administration via a different route. When the relative bioavailability is measured as compared to an intravenously administered drug, the following formula may be used:

$$relative bioavailability = \frac{{[ AUC ]}_{A} * {dose}_{B}}{{[ AUC ]}_{B} * {dose}_{A}}$$

The term “danazol” used herein refers in a non limiting manner, to Azol, Cyclomen, Danocrine and Danol

The term “endometriosis” is herein referred to as including endometriosis, pelvic endometriosis and adenomyosis. Symptoms of endometriosis may depend on the site of active endometriosis. Its main symptom is pelvic pain in various manifestations. Symptoms often worsen with the menstrual cycle. Symptoms of endometriosic-related pain may include, but are not limited to:

dysmenorrhea—painful, sometimes disabling cramps during menses; progressive pain, that may include lower back pains linked to the pelvis.
chronic pelvic pain—typically accompanied by lower back pain or abdominal pain
dyspareunia—painful sex.
dysuria—urinary urgency, frequency, and sometimes painful voiding.

It is herein acknowledged that throbbing, gnawing, and dragging pain to the legs are reported more commonly by women with endometriosis.

The term “menorrhagia” used herein refers to excessive bleeding during menstruation. More specifically, it is an abnormally heavy and prolonged menstrual period at regular intervals. The main causes of menorrhagia include abnormal blood clotting, disruption of normal hormonal regulation of periods or disorders of the endometrial lining of the uterus. It may be associated with abnormally painful periods, also refers to as dysmenorrhea.

The term “Dysmenorrhea” or “dysmenorrhoea” used herein refers to a gynecological medical condition characterized by severe uterine pain during menstruation. Dysmenorrhea is diagnosed when the pain is so severe as to limit normal activities, or require medication.

The term “dissolution” used herein refers to dissolving, liquefaction, melting, deliquescence, breaking up, decomposition, disintegration or any combination thereof. In accordance with some embodiments of the invention, the aforementioned term refers to dissolution of a polymer in a solvent.

The term ‘immediate release’ used herein refers to the effervescent properties of the vaginally administrable composition, having a disintegration time of less than about 3 minutes in water at room temperature or less than about 15 minutes in the vagina.

The term “surfactant” is herein broadly referred to a material that can significantly reduce the surface tension of water when used in very low concentrations. Surfactants may act as detergents, wetting agents, emulsifiers, foaming agents, and dispersants. Surfactants are usually classified based on their dissociation in water. It is within the scope of the present invention that the surfactant used in the vaginally administrable composition is selected from a group comprising a hydrophilic surfactant, a non hydrophilic surfactant, a polymeric surfactant and mixtures thereof. More specifically, the aforementioned surfactant suitable in this pharmaceutical composition may include anionic, cationic, ionic, non ionic and zwitterionic surfactants. An example of a solubilizing component is a d-alpha-tocopheryl polyethylene glycol 1000 succinate.

According to some embodiments of the present invention, the pharmaceutical composition is formulated as a solid carrier or as a thin film delivery form.

According to a further embodiment of the invention, the solid carrier includes a substrate and an encapsulation coat on the substrate. The encapsulation coat may include a hydrophilic surfactant. Optionally, the encapsulation coat can include a pharmaceutical active ingredient, an ionic or non-ionic hydrophilic surfactant, or both a pharmaceutical active ingredient and a hydrophilic surfactant.

The composition of the present invention or alternatively the substrate of the aforementioned composition can be in a form of a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule.

In certain embodiments of the invention, a powder constitutes a finely divided i.e. milled, micronized, nanosized, precipitated, form of an active ingredient or additive molecular aggregates or a compound aggregate of multiple components or a physical mixture of aggregates of an active ingredient and/or additives.

According to a further embodiment of the invention, the vaginally administrable danazol, at least one NSAID, or a danazol combined with NSAID compositions may additionally contain a bioadhesive polymer adapted to increase the adhesiveness of the composition to a vaginal mucosal surface. This property of the composition of the present invention is specifically adapted for the administration of the solid composition in heavy menstrual bleeding conditions, such that effective doses of danazol and NSAID are delivered to the target tissue.

The term “bioadhesive polymer” or “bioadhesive agent” or “bioadhesive excipient” used herein refers in a non limiting manner to polymers that tend to have bioadhesive properties. Hydrophilic polymers, such as poly [acrylic acid], PEG (Polyethylene glycol), that contain carboxylic groups tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, for example POLYOX™ while others are hydrogels. The at least one bioadhesive polymer may comprise any mixture of the above components.

It is further within the scope of the present invention that the bioadhesive agent used in the vaginal composition is hyaluronic acid, naturally present in the human body.

According to a further embodiment of the invention, the vaginally administrable composition may additionally contain an acidic agent. The acidic agent is preferably adapted to increase the stability in the vagina after vaginally applying the composition. It is noted that the stability of the composition increases in acidic pH and decreases in alkaline medium, especially at pH above 9.0 (Gadkariem et al, 2003). Thus it is herein disclosed that according to some embodiments of the invention, the composition may contain an acidic pH agent, adapted to increase the stability of the danazol component in the carrier and within the vagina over an extended period of time.

The term “acidifying agent” used herein may include a citric acid, a lactic acid, an adipic acid, boric acid, acetic acid, glacial acetic acid, citric acid, fumaric acid, hydrochloric acid, diluted hydrochloric acid, malic acid, nitric acid, phosphoric acid, sulfuric acid and tartaric acid, acidic buffer, mild organic acids approved for pharmaceutical applications or any combination thereof.

According to another core aspect of the invention the vaginally administrable composition additionally comprises a protective agent. In one embodiment, the protective agent is formulated in an extended release form to provide protection against at least one of bacterial, viral or fungal infection of the vagina over a prolonged period of time. In a different embodiment the protective agent is formulated in an immediate release form.

In a further embodiment, the protective agent may also include a probiotic agent such as probioic bacteria suitable for the vaginal flora.

In another embodiment, the pharmaceutical composition being formed of different combinations of pharmaceutical active ingredients, hydrophilic surfactants, lipophilic surfactants and triglycerides.

Thus, it is also within the scope of the present invention that the vaginally administrable composition may possess dual therapeutic effects both on treatment and prevention of endometriosis, adenomyosis, mennorhagia, dysmenorrhea and menstruation pains and on protection against vaginal infection and sexually transmitted diseases (STDs).

According to another embodiment of the invention, the protective agent has an acidic pH.

The term “protective agent” used herein refers to a substance which is adapted to provide protection against vaginal infection cased by bacterial, fungal and viral agents and parasites. More specifically, the vaginally administrable composition may additionally contain a protective agent selected from a group comprising antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones and biochemical intermediates, or a mixture thereof.

According to a further embodiment of the invention, the vaginally administrable composition may additionally contain an anti androgen agent adapted to prevent androgenic biological side effects. The anti androgen agent may be administered to the patient vaginally or orally.

The aforementioned androgenic biological side effects may include thickening of the body's non-reproductive tract tissues, such as the skeletal muscles, bones, the larynx and vocal chords and a decrease in body fat. Androgenic steroid effects also include the growth of the male reproductive tract and the development of male secondary sexual characteristics.

The term “antiandrogen” or “anti androgenic agent” used herein refers to an androgen antagonist, which is any of a group of hormone receptor antagonist compounds that are capable of preventing or inhibiting the biologic effects of androgens [1] on their responsive tissues in the body. Anti androgens mode of action is usually based on blocking the appropriate receptors, competing for binding sites on the cell's surface, obstructing the androgens' pathway. Currently available anti androgen drugs include: Spironolactone (Aldactone, Spiritone), a synthetic 17-spirolactone corticosteroid, which is a renal competitive aldosterone antagonist in a class of pharmaceuticals called potassium-sparing diuretics, used primarily to treat low-renin hypertension, hypokalemia, and Conn's syndrome; Cyproterone acetate (Androcur, Climen, Diane 35, Ginette 35), a synthetic steroid, a potent antiandrogen that also possesses progestational properties; Flutamide (Eulexin), nilutamide (Anandron, Nilandron) and bicalutamide (Casodex), nonsteroidal, pure antiandrogens. Flutamide is the oldest and has more unwanted side-effects than the others. Bicalutamide is the newest and has the least side-effects; Ketoconazole (Nizoral), an imidazole derivative used as a broad-spectrum antifungal agent effective against a variety of fungal infections, side-effects include serious liver damage and reduced levels of androgen from both the testicles and adrenal glands; Ketoconazole is a relatively weak antiandrogen; Finasteride (Proscar, Propecia) and dutasteride (Avodart), inhibitors of the 5-α-reductase enzyme that prevent the conversion of testosterone into dihydrotestosterone (DHT). Finasteride blocks only 5-α-reductase type II, dutasteride also blocks type I. They are not general antiandrogens in that they do not counteract the effects or production of other androgens other than DHT; however, DHT is 3-5 times more potent than testosterone or other androgens (except in skeletal muscle tissue, where testosterone is the main androgen); Bexlosteride; Izonsteride; Epristeride and Turosteride or any mixture thereof.

As used herein, the term “foam” or “mousse” is herein defined as any lightweight material in cellular form which is made by introducing gas bubbles into a liquid phase.

As used herein, the term “foam forming agent” is meant to include foam producing agents and compounds that are able to generate a foamable composition when admixed with a liquid or gel composition. The foamable composition generates a foam within a dispensing device or upon dispensing from the dispensing device. In certain embodiments the present invention provides a stable foam, i.e. wherein breaking of the foam is delayed.

The foam base formulations offer an improved compliancy, i.e. the foam form could be applied infrequently to the treated area (e.g. once/twice daily). The foam-based formulation provides an improved administration to the treated subject, since foam does not require special position. Furthermore, formulation in the form of a foam enable improved delivery of the medicament, so as the foam evaporates spontaneously after pre-determined period of time (by formulation), out of the target tissue without having residuals. When the foam is applied to the vagina, it does not leave any residue, stains or odor after it dries. Moreover, the uniqueness of the foam formulation is that there is a relatively uniform concentration of active ingredients at every site of the foam surface, hence the contact area of the active ingredient within the vaginal cavity is effectively increased. The foam formulation further enables the active therapeutic agent/s to contact rapidly the treated area with substantially 100% coverage, and can improve penetration into the affected area. Among other advantages, use of a foam-based formulation avoids the risk of penetration or protrusion of a solid carrier or device to the vaginal tissue walls. Another advantage is in the measured, dosing accompanied by the delivery device. A premeasured dose obviates the uncertainties associated with delivering the correct amount of medicament.

As used herein, the term “pharmaceutical composition” or “medicament” or “therapeutically active agent” or “active agent” or “agent” or “active ingredient”, are all broadly used to mean any chemical or material that is desired to be applied, administered or used to treat gynecological conditions or disorders and can include, by way of illustration and not limitation, any substance which is capable of altering a biologic, physiologic and/or immunologic function, and also substances generally referred to as pharmacological agents and drugs, including antibiotic agents, antibacterial agents, antifungal agents, steroid agents, anti-inflammatory agents and local anesthetic agents. The invention is meant to include a pharmaceutical composition comprising at least one therapeutically active agent, particularly at least one NSAID molecule or compound, danazol and a combination thereof.

The term ‘about’ used hereinafter refers to a range of 10% below or above the referred value.

The term “add-on” formulation′ or “add-on” to a tampon formulation′ used hereinafter refers to solid formulations that can be configured as a layer or film at least partially coating a substrate such as a tampon, adapted for vaginal administration. In preferred embodiments, the thin layer or film has immediate release properties. Upon contact with the vaginal epithelium mucosal tissue and/or menstrual fluid or other vaginal discharges effective therapeutic doses of the at least one active ingredient (i.e. at least one NSAID and/or danazol) rapidly disintegrate from the layer or film at least partially coating the substrate to the target tissue, namely the vaginal or endometrial tissue.

Compositions according to the present invention may comprise any conventional carriers, excipients or adjuvant used in pharmaceuticals, personal care formulations and compositions or veterinary formulations.

It is within the scope of the present invention that the vaginally administrable composition may contain micronized danazol. Such micronized particles are adapted to increase the surface area of the pharmaceutical carrier and therefore improve dissolution and bioavailability of the drug.

In accordance with a further embodiment of the invention, the vaginally administrable composition comprises at least one pharmaceutically acceptable non-effervescent excipient, polymer, copolymer and/or carrier. The excipients may include diluents, binders, surfactants, polymers, copolymers, polysaccharides or granulating agents, glidants (flow aids) and lubricants to ensure efficient tabletting; and, disintegrants. A polymer coating is often applied to make the carrier smoother, to control the release rate of the active ingredient, to make it more resistant to the environment (extending its shelf life), and/or to enhance the carrier's appearance. In addition such excipients may include pigments to make the carrier visually attractive.

In accordance with a further embodiment of the invention, the non-effervescent excipient is further selected from a group comprising Hydroxypropyl methyl cellulose (HPMC), starch, kollidone, ethanol, Klucel, Ethocel, FMC nanoparticles, Carbopol, Lactose, Magnesium Stearat, Silicon dioxide, lipidic carriers, gums, cellulose derivatives and mixtures thereof.

In accordance with a further embodiment of the invention, the vaginally administrable composition comprises a polymer which is further selected from a group consisting essentially of Hydroxypropyl methyl cellulose (HPMC) K15M, HPMC LV50 K15M, LV50 (Colorcon), hypromellose polymers, poly ethylene oxide polymers such as polyox, Polyvinylpyrrolidone (PVP), Polyvinylpolypyrrolidone (PVPP), Kollidon SR and mixture thereof.

In accordance with a further embodiment of the invention, the vaginally administrable composition comprises a copolymer comprises polylactic and polyglycolic acids in a predetermined ratio.

In accordance with a further embodiment of the invention the copolymer is poly (lactic-co-glycolic acid) (PLGA) or Eudragit acrylate polymer.

According to another embodiment of the invention, the pharmaceutical composition comprises polymers selected from a group consisting essentially of HPMC K15M, HPMC LV50 K15M, LV50 (i.e. Colorcon). The viscosity grades definitions of the aforementioned polymers relate to viscosities of aqueous solutions 2% w/w of hypromellose polymers (for example 50 centipoise and 15000 centipoise).

According to a preferred embodiment of the invention, the pharmaceutical composition comprises polyox as a major polymer, or other polymers such as Polyvinylpyrrolidone (PVP), which is a water-soluble polymer made from the monomer N-vinylpyrrolidone or Polyvinylpolypyrrolidone (PVPP) (crospovidone), which is a highly cross-linked modification of PVP.

The term “polyox” used herein refers in a non limiting manner to POLYOX™ Water-Soluble Resins, which are non-ionic poly (ethylene oxide) polymers. These are hydrophilic polymers available in a wide range of molecular weights. Polyox resins are suitable for drug delivery systems such as the present invention, as they provide a number of benefits including: wide range of molecular weights which enable formulation flexibility, direct compression and granulation, rapid hydration and swelling, and rapid gel formation. POLYOX™ meets the requirements of the United States Pharmacopoeia (USP) and compliance with US Food Chemicals Codex.

According to another embodiment of the invention, the vaginally administrable composition comprises PLGA. The term “PLGA” used herein refers to poly (lactic-co-glycolic acid), a copolymer of polylactic polyglycolic acids in predetermined ratios which control their physical characteristics and degradation rates. PLGA is a biodegradable and biocompatible polymer, and therefore approved by the Food and Drug Administration (FDA) for uses in therapeutic devices. PLGA is synthesized by means of random ring-opening co-polymerization of two different monomers, the cyclic dimers (1,4-dioxane-2,5-diones) of glycolic acid and lactic acid. Common catalysts used in the preparation of this polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product.[1] Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid). PLGAs are amorphous rather than crystalline and show a glass transition temperature in the range of 40-60° C. Unlike the homopolymers of lactic acid (polylactide) and glycolic acid (polyglycolide) which show poor solubilities, PLGA can be dissolved by a wide range of common solvents, including chlorinated solvents, tetrahydrofuran, acetone or ethyl acetate.

According to another embodiment of the present invention, the vaginally administrable composition comprises Hypromellose polymers (INN), also referred to as hydroxypropyl methylcellulose (HPMC), which is a semisynthetic, inert, viscoelastic polymer. Hypromellose in an aqueous solution, and unlike methylcellulose, it does not exhibit thermal gelation property. When the solution heats up to a critical temperature, the solution congeals into a non-flowable but semi-flexible mass. Typically, this critical (congealing) temperature is inversely related to both the solution concentration of HPMC and the concentration of the methoxy group within the HPMC molecule. The higher the concentration of the methoxy group within the HPMC molecule, the lower the critical temperature is. The inflexibility/viscosity of the resulting mass, however, is directly related to the concentration of the methoxy group (the higher is the concentration, the more viscous or less flexible is the resulting mass). According to certain embodiments, typical viscosity test will specify the following: Solution concentration (1%, 2%, 1.9% bone dry, etc.), Viscometer (Brookfield LV or RV, Hoppler falling ball, Haake Rotovisco, etc.), Viscometer spindle number (1˜4 for Brookfield LV, 1˜7 for Brookfield RV, etc.), Solution Temperature (20° C., 25° C., etc.), Degree of substitution which is the average level of methoxy substitution on the cellulose chain.

According to a further embodiment of the present invention, the HPMC polymers specifically relate to colorcon polymers. Such polymers provide complete film coating systems, modified release technologies, and functional excipients. According to another embodiment of the invention, the pharmaceutical composition comprises Kollidon SR. The term “Kollidon SR” used herein refers to a blend of polyvinyl acetate and povidone for use as a matrix-forming agent in directly compressible sustained—release tablets. It consists of 80% polyvinyl acetate and 19% povidone along with small quantities of sodium lauryl sulfate and silica as stabilizers. According to certain embodiments, Kollidon SR is used in formulations of pH independent sustained release matrix dosage forms such as tablets, pellets and granules by either, direct compression, roller compaction, wet granulation or extrusion.

According to another embodiment, the pharmaceutical composition of the present invention comprises EUDRAGIT® RL 100, which is a copolymer of ethyl acrylate, methyl methacrylate and a low content of methacrylic acid ester with quaternary ammonium groups. In some embodiments, the ammonium groups are present as salts and make the polymers permeable.

According to a further embodiment of the invention, the vaginally administrable composition is provided in a solid dosage form selected from a group comprising a tablet, caplet, a capsule, a lozenge, a dragee, a suppository, a wafer, a tab, a bar, a stick, a granule, a bead, a layer, a strip, a film, a particle, nanoparticles, extrusion/spheronization particles, a matrix or any combination thereof.

According to a further embodiment of the invention the vaginally administrable composition is formulated in micronized particles or powder form.

According to a further embodiment of the invention the vaginally administrable composition is administered vaginally once or twice per day. In other embodiments the composition is administered one to six times per day.

According to a further embodiment of the invention, the vaginally administrable composition is additionally provided with an applicator or dispenser suitable for vaginal administration.

The present invention further provides a kit useful for treating gynecological disorders, especially at least one of endometriosis, adenomyosis, mennorhagia, dysmenorrhea and menstruation pains, wherein said kit comprising: (a) a plurality of solid dosage forms, each solid dosage form containing (i) effective amount of at least one active ingredient selected from a group consisting of danazol, at least one non steroidal anti inflammatory drug (NSAID) and a combination thereof, (ii) at least one pharmaceutically acceptable carrier or excipient, wherein said composition is adapted to be vaginally administrable further wherein said composition is in an immediate release form (b) an applicator suitable for vaginal administration; and, (c) instructions for use of said composition and said applicator.

In a core aspect of the invention, the composition has a rapid disintegration time in the vagina. The kit preferably comprises an applicator for vaginal administration.

Reference is now made to the kit as defined above, wherein said carrier or excipient is selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

Reference is now made to the kit as defined above, wherein the composition comprises between about 5 to about 12 wt. % effervescent excipient.

Reference is now made to the kit as defined above, wherein said composition is formulated in a form selected from a group consisting of a solid dosage form, foam and a mousse.

Reference is now made to the kit as defined above, wherein said composition is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

Reference is now made to the kit as defined above, wherein said at least one Thin Film type or PharmFilm type or mucoadhesive type excipient is selected from a group consisting of: hydroxypropyl methyl cellulose (HPMC) K4M, Carbopol 934, hyaluronic acid, Gelatin, Mannitol, Citric acid, Chitosan, Sodium Alignate, Cyclodextrin and combination thereof.

Reference is now made to the kit as defined above, wherein said at least one FDT type excipient is selected from a group consisting of: Mannitol, Lactose, Erythritol, Xylitol, Glucose, Sucrose, Sorbitol, Maltitol, Trehalose, Maltose and mixtures thereof.

Reference is now made to the kit as defined above, wherein said bioadhesive type agent is selected from a group consisting of Carbopol 971, hyaluronic acid, HPMC, hydrophilic polymers such as polyox, PEG (Polyethylene glycol), hydrogels, cellulose derivatives such as, sodium alginate, carboxymethylcellulose, hydroxymethylcellulose, methylcellulose, hydroxypropyl methyl cellulose (HPMC) K4M, Carbopol 934, Gelatin, Mannitol, Citric acid, Chitosan, Sodium Alignate, Cyclodextrin and any mixture thereof.

Reference is now made to the kit as defined above, wherein said probiotic agent is selected from a group consisting of lactic-acid bacteria such as Lactobacillus rhamnosus, bifidobacterium, streptococcus, factors that stimulate the growth of protective organisms, enzymes, vitamins and cellular factors and mixtures thereof.

Reference is now made to the kit as defined above, wherein said acidifying agent is selected from a group consisting of boric acid, acetic acid, glacial acetic acid, fumaric acid, diluted hydrochloric acid, citric acid, lactic acid, malic acid, nitric acid, phosphoric acid, sulfuric acid, tartaric acid, acidic buffer and mild organic acids approved for pharmaceutical applications and any combination thereof.

Reference is now made to the kit as defined above, wherein said surfactant is selected from a group consisting of: a hydrophilic, a non hydrophilic, a polymeric, an ionic, a non ionic and zwitterionic surfactant.

Reference is now made to the kit as defined above, wherein said protective agent is selected from a group consisting of antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones, biochemical intermediates, and any combination thereof.

Reference is now made to the kit as defined above, wherein said dispersing agent is selected from a group consisting of a surfactant, a cholesteryl ester, a fatty acid, a phospholipid, a carbohydrate, a protein and a mixture thereof.

Reference is now made to the kit as defined above, wherein the surfactant is selected from a group consisting of a natural ionic surfactant, a synthetic ionic surfactant, a natural non-ionic surfactant, a synthetic non-ionic surfactant and a mixture thereof.

Reference is now made to the kit as defined above wherein the solid dosage form is formulated in a fast dissolving tablet (FDT) type formulation.

Reference is now made to the kit as defined above, wherein the solid dosage form further comprises at least one excipient adapted for thin film or mucoadhesive drug delivery.

Reference is now made to the kit as defined above, wherein the composition comprises micronized danazol.

Reference is now made to the kit as defined above, wherein the composition is in a dosage form which can be provided in the form of a minicapsule, a capsule, a tablet, an implant, a troche, a lozenge, a minitablet, a temporary or permanent suspension, an ovule, a suppository, a wafer, a chewable tablet, a quick or fast dissolving tablet, an effervescent tablet, a buccal-type or sublingual-type solid, a granule, a film, a sprinkle, a pellet, a bead, a pill, a powder, a triturate, a platelet, a strip or a sachet.

Reference is now made to the kit as defined above, wherein said at least one NSAID is selected from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt or derivative thereof and any combination thereof.

Reference is now made to the kit as defined above, wherein said at least one NSAID is further selected from a group consisting of: aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, and a pharmaceutically acceptable salt or derivative thereof and any combination thereof.

Reference is now made to the kit as defined above, wherein the solid carrier unit is administered vaginally once to six times per day.

The present invention further provides a composition for vaginal administration, prepared by steps of: (a) preparing a mixture comprising an effective amount of at least one active ingredient selected from a group consisting of danazol, an effective amount of at least one NSAID and a combination thereof, and at least one pharmaceutically acceptable carrier or excipient and, (b) forming said composition in an immediate release form.

Reference is now made to a composition for vaginal administration prepared by steps as defined above, wherein the composition is formed in a solid dosage form, foam or mousse type delivery form.

Reference is now made to a composition for vaginal administration prepared by steps as defined above, wherein the composition is formed in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

The present invention further provides a danazol and/or danazol combined with at least one NSAID composition for vaginal administration, prepared by steps of: (a) preparing a mixture comprising an effective amount of at least one active ingredient selected from a group consisting of danazol, an effective amount of at least one NSAID and a combination thereof, and at least one pharmaceutically acceptable carrier or excipient and, (b) forming a solid carrier by compaction of the mixture into the solid carrier.

The present invention further provides a danazol and/or danazol combined with at least one NSAID composition for vaginal administration, prepared by steps of: (a) preparing a mixture comprising an effective amount of at least one active ingredient selected from a group consisting of danazol, an effective amount of at least one NSAID and a combination thereof, and optionally at least one effervescent excipient, and, (b) forming a solid carrier by compaction of the mixture into the solid carrier.

Reference is now made to a composition for vaginal administration prepared by steps as defined above, wherein the composition is in an immediate release form.

Reference is now made to a composition for vaginal administration prepared by steps as defined above, wherein the composition has a rapid disintegration time in the vagina.

Reference is now made to a composition for vaginal administration prepared by steps as defined above, wherein the mixture is further prepared by steps of a wet granulation process.

Reference is now made to a composition for vaginal administration prepared by steps as defined above, wherein the mixture is further prepared by steps of: (a) preparing a first mixture comprising the effective amount of danazol or at least one NSAID or a combination thereof and optionally at least one pharmaceutically accepted carrier or excipient; (b) preparing a second mixture by adding water to the first mixture; (c) granulating the second mixture; (d) drying the granulation mixture; and, (e) compressing the granules so as to form the solid carrier composition.

The present invention further provides a composition adapted to be vaginally administrable useful for treating gynecological disorders, especially endometriosis, adenomyosis, mennorhagia, dysmenorrhea and menstruation pains, prepared by steps of: (a) preparing a first mixture containing a polymeric solution comprising at least one polymer and at least one solvent; (b) preparing a second mixture by adding said danazol or said at least one NSAID or a combination thereof to said first mixture.

In a further embodiment, the method as defined above further comprises steps of preparing a thin film type form by casting said second mixture into predetermined containers (petri dishes) and allowing it to dry.

Reference is now made to the composition for vaginal administration prepared by steps as defined above, wherein the composition is further prepared by steps of adding at least one of Carbopol solution and glycerine to said first mixture.

Reference is now made to the composition for vaginal administration prepared by steps as defined above wherein the composition is further prepared by steps of cutting the dried film into predetermined units.

Reference is now made to the composition for vaginal administration prepared by steps as defined above wherein the composition is further prepared by steps of storing said units in desiccation conditions until use.

Reference is now made to a composition for vaginal administration prepared by steps as defined above, wherein the mixture additionally comprises an effervescent excipient.

Reference is now made to a composition for vaginal administration prepared by steps as defined above, wherein the mixture additionally comprises a lubricating agent, such as magnesium stearate.

Reference is now made to a composition for vaginal administration prepared by steps as defined above, wherein the mixture comprises between about 10 mg to about 250 mg of danazol.

Reference is now made to a composition for vaginal administration prepared by steps as defined above, wherein the mixture comprises between about 50 mg to about 300 mg of the at least one NSAID ingredient.

Reference is now made to a composition for vaginal administration prepared by steps as defined above, wherein the NSAID compound is selected from a group comprising Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt thereof and any combination thereof.

Reference is now made to a composition for vaginal administration prepared by steps as defined above, wherein said at least one NSAID is further selected from a group essentially consisting of: aspirin, ibuprofen, naproxen, Diclofenac, Cox-2 inhibitors, etodolac, indomethacin, ketoprofen, piroxicam, folmetin, diclofenac, piroxicam, tenoxicam, mecoxicam, meloxicam, mefenamic acid, ibufenac, ketoprofen, and a pharmaceutically acceptable salt thereof and any combination thereof.

Reference is now made to a composition for vaginal administration prepared by steps as defined above, wherein said composition is further prepared by steps of incorporating into said mixture at least one excipient or diluent or carrier selected from a group essentially consisting of effervescent excipient, mucoadhesive type excipient, FDT type excipient, bioavailability enhancing agent or excipient, a surfactant, a bioadhesive excipient, acidic agents, diluents, binders, granulating agents, glidants, lubricants, disintegrates, dissolving agents, solubilising agents, bioadhesive agents, polysaccharides, polymers, copolymers and mixtures thereof.

Reference is now made to a composition for vaginal administration prepared by steps as defined above, wherein said mucoadhesive or thin film delivery excipient is selected from a group consisting essentially of: hydroxypropyl methyl cellulose (HPMC) K4M, Carbopol 934, Carbopol 971, Hyaluronic Acid, Gelatin, Mannitol, Citric acid, Chitosan, Sodium Alignate, Cyclodextrin and combination thereof.

Reference is now made to a composition for vaginal administration prepared by steps as defined above, wherein said at least one FDT type excipient is selected from a group consisting essentially of: Mannitol, Lactose, Erythritol, Xylitol, Glucose, Sucrose, Sorbitol, Maltitol, Trehalose, Maltose and mixtures thereof.

Reference is now made to a composition for vaginal administration prepared by steps as defined above, wherein the composition is further prepared by steps of incorporating into the mixture at least one bioavailability enhancing agent selected from a group comprising a surfactant adapted to increase danazol solubility in the vagina, a bioadhesive polymer adapted to increase adhesiveness of the composition to a vaginal mucosal surface, an acidifying agent, or any combination thereof.

The present invention further provides a method for treating gynecological condition, especially endometriosis, adenomyosis, dysmenorrhea, mennorhagia and menstruation pains, wherein said method comprises steps of: (a) preparing a composition comprising at least one active ingredient selected from a group consisting of at least one NSAID, danazol, and a combination thereof, and at least one carrier or excipient; and, (b) administering said composition vaginally at a therapeutically effective dosage.

Reference is now made to the method as defined above. The aforementioned method comprising an additional step of formulating said composition as a solid dosage form, foam or mousse type delivery form.

Reference is now made to the method as defined above. The aforementioned method comprising an additional step of formulating said composition in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

Reference is now made to the method as defined above. The aforementioned method comprising an additional step of selecting said carrier or excipient from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

The present invention further provides a method for treating a gynecological condition, especially endometriosis, adenomyosis, dysmenorrhea, mennorhagia and menstruation pains, wherein the method comprises steps of: (a) preparing a composition comprising at least one active ingredient selected from a group consisting of at least one NSAID, danazol, and a combination thereof, and at least one carrier or excipient; (b) administering said composition vaginally at a therapeutically effective dosage.

The present invention further provides a method for treating gynecological disorders, especially dysmenorrhea, mennorhagia and menstruation pains, comprising steps of: (a) formulating a composition in a solid dosage form comprising at least one non steroidal anti inflammatory drug (NSAID); and, (b) administering said solid dosage form vaginally at a therapeutically effective dosage.

Reference is now made to the method as defined above. The aforementioned method further comprises steps of administering said composition vaginally thereby reducing menstrual blood loss by about 10%, particularly by about 10% to about 30%.

Reference is now made to the method as defined above. The aforementioned method further comprises steps of administering said composition vaginally thereby reducing menstrual blood loss by at least about 30%.

Reference is now made to the method as defined above. The aforementioned method further comprises steps of selecting said at least one NSAID from a group consisting essentially of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors and a pharmaceutically acceptable salt thereof and any combination thereof.

Reference is now made to the method as defined above. The aforementioned method further comprises steps of selecting said at least one NSAID from a group consisting essentially of: aspirin, ibuprofen, naproxen, Diclofenac, Cox-2 inhibitors, etodolac, indomethacin, ketoprofen, piroxicam, folmetin, diclofenac, piroxicam, tenoxicam, mecoxicam, meloxicam, mefenamic acid, ibufenac, ketoprofen, and a pharmaceutically acceptable salt thereof and any combination thereof.

Reference is now made to the method as defined above. The aforementioned method further comprises steps of administering said composition in a daily dosage of between about 5 mg to about 2000 mg of said at least one NSAID.

Reference is now made to the method as defined above. The aforementioned method further comprises steps of administering said composition in a dosage unit of between about 5 mg to about 500 mg of said at least one NSAID.

Reference is now made to the method as defined above. The aforementioned method further comprises steps of administering said composition vaginally one to six times per day.

The present invention further provides a method for treating gynecological disorders, especially endometriosis, adenomyosis, dysmenorrhea, mennorhagia and menstruation pains, wherein said method comprises steps of: (a) formulating a composition comprising at least one active ingredient selected from a group consisting of at least one NSAID, danazol and a combination thereof, and at least one pharmaceutically accepted carrier or excipient adapted for immediate release delivery type form; and, (b) administering said composition vaginally at a therapeutically effective dosage.

Reference is now made to the method as defined above. The aforementioned method further comprises steps of formulating said composition in an immediate or rapid release form.

Reference is now made to the method as defined above. The aforementioned method further comprises steps of formulating said composition with at least one carrier or excipient or diluent.

Reference is now made to the method as defined above. The aforementioned method further comprises steps of formulating said composition with at least one effervescent excipient.

Reference is now made to the method as defined above. The aforementioned method further comprises steps of formulating said composition in a form selected from a group consisting of a solid dosage form, foam and a mousse.

Reference is now made to the method as defined above. The aforementioned method further comprises steps of formulating said composition in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

Reference is now made to the method as defined above. The aforementioned method further comprises steps of formulating said composition with a carrier or excipient selected from a group consisting of diluents, binders, granulating agents, glidants, lubricants, surfactants, disintegrates, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

Reference is now made to the method as defined above. The aforementioned method further comprises steps of formulating said composition in a fast dissolving tablet (FDT) type form.

Reference is now made to the method as defined above. The aforementioned method further comprises steps of formulating said composition in a thin film type or mucoadhesive type formulation.

Reference is now made to the method as defined above. The aforementioned method further comprises steps of formulating said composition in an “add-on” to a substrate, especially a tampon formulation, configured for vaginal application.

Reference is now made to the method as defined above. The aforementioned method further comprises steps of formulating said composition with at least one excipient adapted for mucoadhesive or thin film type delivery.

Reference is now made to the method as defined above. The aforementioned method further comprises steps of selecting said at least one excipient adapted for thin film type or mucoadhesive delivery from a group consisting essentially of: hydroxypropyl methyl cellulose (HPMC) K4M, Carbopol 934, Carpobol 971, Hyaluronic acid, Gelatin, Mannitol, Citric acid, Chitosan, Sodium Alignate, Cyclodextrin and combination thereof.

Reference is now made to the method as defined above. The aforementioned method further comprises steps of selecting said at least one FDT type excipient from a group consisting essentially of: Mannitol, Lactose, Erythritol, Xylitol, Glucose, Sucrose, Sorbitol, Maltitol, Trehalose, Maltose and mixtures thereof.

Reference is now made to the method as defined above. The aforementioned method further comprises steps of administering said composition in a daily unit dosage of between about 10 mg to about 200 mg of danazol.

Reference is now made to the method as defined above. The aforementioned method further comprises steps of administering said composition in a daily unit dosage of less than about 10 mg of danazol.

Reference is now made to the method as defined above, wherein the composition has a rapid disintegration time in the vagina.

Reference is now made to the method as defined above. The aforementioned method further comprising steps of administering the composition such that the absolute bioavailability ratio of danazol and/or the at least one NSAID in the bloodstream is lowered as compared to conventional oral and/or conventional vaginal composition comprising comparable amounts of danazol, and as compared to comparable amounts of NSAID administered orally.

Reference is now made to the method as defined above. The aforementioned method further comprising steps of administering the composition such that the relative bioavailability ratio of danazol and/or the at least one NSAID compound at the endometrial and/or vaginal tissue is higher as compared to conventional oral and/or conventional vaginal composition comprising comparable amounts of danazol or comparable amounts of NSAID administered orally.

Reference is now made to the method as defined above. The aforementioned method further comprising steps of administering the composition such that the ovarian and endometrial danazol concentrations are comparable to those of after daily oral administration of about four times higher dosage of danazol.

Reference is now made to the method as defined above. The aforementioned method further comprising steps of administering the composition such that the level of danazol and at least one NSAID in the serum or bloodstream in non detectable.

Reference is now made to the method as defined above. The aforementioned method further comprising steps of providing the composition with an applicator or dispensing device suitable for vaginal administration.

Reference is now made to the method as defined above. The aforementioned method additionally comprises steps of preparing the composition by steps of: (a) preparing a mixture comprising an effective amount of danazol, at least one NSAID, or a combination thereof and at least one pharmaceutically acceptable carrier or excipient, (b) forming the compaction in an immediate release form.

Reference is now made to the method as defined above. The aforementioned method additionally comprises steps of preparing the composition in the form of a solid carrier by steps of: (a) preparing a mixture comprising an effective amount of danazol, or at least one NSAID, or a combination thereof, and at least one pharmaceutical accepted excipient or carrier, and at least one effervescent excipient; and, (b) forming a solid carrier by compaction of the mixture into the solid carrier.

Reference is now made to a use of a vaginally administrable composition, in the manufacture of a medicament for the treatment of a gynecological disorder, especially endometriosis, adenomyosis, menorrhagia and menstruation pains. The aforesaid composition comprising an active ingredient selected from a group consisting of an effective amount of danazol, at least one NSAID, and a combination thereof, and at least one excipient or carrier, wherein the composition has a rapid disintegration time in the vagina.

Reference is now made to a use of a vaginally administrable composition, in the manufacture of a medicament for the treatment of a gynecological disorder, especially endometriosis, adenomyosis, menorrhagia and menstruation pains. The aforesaid composition comprising an active ingredient selected from a group consisting of an effective amount of danazol, at least one NSAID, and a combination thereof, and optionally at least one effervescent excipient, wherein the composition has a rapid disintegration time in the vagina.

Reference is now made to a use of a vaginally administrable composition for the treatment of a gynecological disorder, especially endometriosis, adenomyosis, menorrhagia, dysmenorrhea and menstruation pains, said composition comprising an effective amount of at least one non steroidal anti inflammatory drug (NSAID), wherein said composition is formulated in an immediate release form.

Reference is now made to a use of a vaginally administrable composition, for the treatment of a gynecological disorder, especially endometriosis, adenomyosis, menorrhagia, dysmenorrhea and menstruation pains, said composition comprising an effective amount of danazol wherein said composition is formulated in an immediate release form.

Reference is now made to a use of a vaginally administrable composition, for the treatment of a gynecological disorder, especially endometriosis, adenomyosis, menorrhagia, dysmenorrhea and menstruation pains, said composition is formulated in a form selected from a group consisting of a solid dosage form, foam and a mousse.

Reference is now made to a use of a vaginally administrable composition, for the treatment of a gynecological disorder, especially endometriosis, adenomyosis, menorrhagia, dysmenorrhea and menstruation pains, said composition is formulated in a solid dosage form selected from a group comprising a tablet, a caplet, a capsule, a lozenge, a dragee, a suppository, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, an “add-on” formulation and any combination thereof.

In order to understand the invention and to see how it may be implemented in practice, a plurality of preferred embodiments will now be described, by way of non-limiting example only, with reference to the following examples.

### Example 1

### Preparation of Immediate Release NSAID Composition for Vaginal Application

Reference is now made to a vaginal composition formulated in an immediate release form, comprising at least one NSAID ingredient as an active compound. Such a composition administered vaginally is effective in treatment of mennorhagia, dysmenorrhea and heavy and prolonged menstruation bleeding and pain symptoms.

The aforementioned vaginal composition contains at least one NSAID compound and at least one excipient for formulating the vaginal composition with immediate release properties.

The NSAID compound may include at least one of aspirin, ibuprofen, naproxen, Diclofenac, Cox-2 inhibitors, etodolac, indomethacin, ketoprofen, piroxicam, folmetin, piroxicam, tenoxicam, mecoxicam, meloxicam, ibufenac, ibuprofen, naproxen, Mefenamic acid, ketoprofen, and a pharmaceutically acceptable salt thereof.

Table 1 below specifies the NSAID compound dose ranges for vaginal administration:

TABLE 1

| NSAID | | Administration |
|---|---|---|
| compound | Dose range | per day |
| Diclofenac | 10-50 mg or | x2/day or |
| | 10-100 mg | x1/day |
| Naproxen | 25-500 mg | x1-2/day |
| Piroxicam | 5-20 mg | x1/day |
| Ibuprofen | 20-200 mg | x1-3/day |
| Indometacin | 5-25 mg | x1-6/day |
| Mefenamic | 25-500 mg | x1-4/day |
| acid | | |

According to one aspect, the vaginal composition preferably contains at least one NSAID compound such as Ibuprofen, Naproxen and Mefenamic acid, in the dosage range of about 50 mg to 300 mg, and optionally at least one of the following inactive ingredients or excipients: Starch, Kollidone CL as a disintegrant, PVP, Lactose, Carbopol or alternatively Hyaluronic acid as a bioadhesive ingredient, polymers such as Polyox and HPMC, Ethanol, Tween 80 as a solubilizer, Syloid F244 as a glidant, Adipic or Lactic acid, Carbomer 934P and Mg Stearate. The vaginal composition may further contain an acidic agent for maintaining an acidic pH in the vagina. Maintaining an acidic pH in the vagina may both contribute to the vaginal health by having an antibacterial effect and might prevent undesirable gelation of the carbopol ingredient which tends to create gels in natural pH. In another embodiment, the vaginal composition may further comprise a coating material such as Opadry II white.

The vaginal composition may also include at least one thin film type or mucoadhesive type excipient such as hydroxypropyl methyl cellulose (HPMC) K4M, Carbopol 934, Gelatin, Mannitol, Citric acid, Chitosan, Sodium Alignate, Cyclodextrin and any combination thereof.

A vaginal composition in a Fast Dissolving Tablet (FDT) type form may further contain at least one FDT excipient selected from a group comprising low compressibility excipients such as Mannitol, Lactose, Erythritol, Xylitol and Glucose, and high compressibility excipients such as Sucrose, Sorbitol, Maltitol, Trehalose, Maltose and mixtures thereof.

It is also within the scope of the present invention that the NSAID vaginal composition further comprises a probiotic component, such as probiotic bacteria, enzymes, vitamins and cellular factors.

A typical vaginal solid dosage form may contain the following ingredients:

| | | |
|---|---|---|
| NSAID compound | 50-300 | mg (i.e. Ibuprofen 100 mg) |
| Lactose anhydrous | 150 | mg |
| Carbopol 971 | 10 | mg |
| Mannitol sprayed dried | 300 | mg |
| Citric acid | 10 | mg |
| Sodium citrate | 7 | mg acidic buffer |
| PVP K30 | 8 | mg |
| Corn starch/starch 1500 | 30 | mg |
| Magnesium Stearate | 5 | mg |

Table 2 below exemplifies a proposed ingredient content and preparation steps of a solid dosage form containing NSAID vaginal composition:

TABLE 2
Ingredients and preparation steps of the vaginal immediate release
composition

| Amount | Ingredients (raw | | Preparation |
|---|---|---|---|
| mg/gr | material) | mg/carrier unit | Part |
| | NSAID | 50-300 | I |
| | | (according to | Wet |
| | | Table 1) | Granulation |
| 141.41 | Starch 1500 | 143.0 | |
| 49.44 | Kollidone CL | 50.0 | |
| 39.56 | PVP 30 cps | 40.0 | |
| 247.22 | Lactose 21A | 250.0 | |
| * | Ethanol, BP | 175.0 | |
| * | Purified Water, | 35.0 | |
| | USP | | |
| 9.89 | Tween 80 | 10.0 | |
| 19.78 | Syloid F244 | 20.0 | II |
| 56.37 | Adipic/Lactic acid | 57.0 | Direct |
| 247.22 | Lactose 30G | 250.0 | compression |
| 9.89 | Mg. Stearate | 10.0 | |
| 14.83 | Carbomer 934P | 15.0 | |
| 8.1 | Opadry II White | 10.0 | III |
| * | Purified Water, | 80.0 | Coating |
| | USP | | |

### Example 2

### Preparation of Immediate Release Danazol with NSAID Composition for Vaginal Application

The vaginal composition preferably contains 10-150 mg of danazol, at least one NSAID compound in the dosage range of about 50 mg to 300 mg (see Table 1), and optionally at least one of the following inactive ingredients or excipients: Starch, Kollidone CL as a disintegrant, PVP, Lactose, Carbopol or alternatively Hyaluronic acid as a bioadhesive ingredient, polymers such as Polyox and HPMC, Ethanol, Tween 80 as a solubilizer, Syloid F244 as a glidant, Adipic or Lactic acid, Carbomer 934P and Mg Stearate. The vaginal composition may further contain an acidic agent for maintaining an acidic pH in the vagina. Maintaining an acidic pH in the vagina may both contribute to the vaginal health by having an antibacterial effect and might prevent undesirable gelation of the carbopol ingredient which tends to create gels in natural pH. In another embodiment, the vaginal composition may further comprise a coating material such as Opadry II white.

The vaginal composition may also include at least one thin film type or mucoadhesive type excipient such as hydroxypropyl methyl cellulose (HPMC) K4M, Carbopol 934, Gelatin, Mannitol, Citric acid, Chitosan, Sodium Alignate, Cyclodextrin and any combination thereof.

The vaginal composition may further contain FDT excipients that may comprise at least one of low compressibility excipients such as Mannitol, Lactose, Erythritol, Xylitol and Glucose, and high compressibility excipients such as Sucrose, Sorbitol, Maltitol, Trehalose, Maltose and mixtures thereof.

It is also within the scope of the present invention that the NSAID vaginal composition further comprises a probiotic component, such as probiotic bacteria, enzymes, vitamins and cellular factors.

A typical vaginal solid dosage form may contain the following ingredients:

| | | |
|---|---|---|
| Danazol | 10-150 | mg |
| NSAID compound | 50-300 | mg (i.e. Ibuprofen 100 mg) |
| Lactose anhydrous | 150 | mg |
| Carbopol 971 | 10 | mg |
| Mannitol sprayed dried | 300 | mg |
| Citric acid | 10 | mg |
| Sodium citrate | 7 | mg acidic buffer |
| PVP K30 | 8 | mg |
| Corn starch/starch 1500 | 30 | mg |
| Magnesium Stearate | 5 | mg |

Table 3 below exemplifies a proposed ingredient content and preparation steps of a solid dosage form containing the danazol and NSAID vaginal composition:

TABLE 3
Ingredients and preparation steps of the vaginal immediate release
composition

| Amount | Ingredients (raw | | Preparation |
|---|---|---|---|
| mg/gr | material) | mg/carrier unit | Part |
| 148.3 | Danazol API | 150.0 | I |
| | NSAID | 50-300 | Wet |
| | | (according to | Granulation |
| | | Table 1) | |
| 141.41 | Starch 1500 | 143.0 | |
| 49.44 | Kollidone CL | 50.0 | |
| 39.56 | PVP 30 cps | 40.0 | |
| 247.22 | Lactose 21A | 250.0 | |
| * | Ethanol, BP | 175.0 | |
| * | Purified Water, | 35.0 | |
| | USP | | |
| 9.89 | Tween 80 | 10.0 | |
| 19.78 | Syloid F244 | 20.0 | II |
| 56.37 | Adipic/Lactic acid | 57.0 | Direct |
| 247.22 | Lactose 30G | 250.0 | compression |
| 9.89 | Mg Stearate | 10.0 | |
| 14.83 | Carbomer 934P | 15.0 | |
| 8.1 | Opadry II White | 10.0 | III |
| * | Purified Water, | 80.0 | Coating |
| | USP | | |

### Example 3

### Preparation of an Effervescent Vaginal NSAID and NSAID Combined with Danazol Composition in the Form of a Solid Carrier

Reference is now made to a vaginal composition containing at least one NSAID compound in the dosage of 50-300 mg or a combination of at least one NSAID compound in the dosage of 50-300 mg with 10-150 mg of danazol, and may further include at least one of the following inactive ingredients: Starch, Kollidone CL as a disintegrant, PVP, Lactose, Ethanol, Tween 80 as a solubilizer, Syloid F244 as a glidant, Adipic or Lactic acid, Carbomer 934P and Mg Stearate. The immediate release vaginal composition, according to the current embodiment, comprises sodium bicarbonate as an effervescent excipient. The vaginal composition may further contain a probiotic compound such as probiotic bacteria, enzymes, vitamins and cellular factors and/or a coating material such as Opadry II white.

In a further embodiment, the vaginal composition may also include at least one thin film type or mucoadhesive type excipient such as hydroxypropyl methyl cellulose (HPMC) K4M, Carbopol 934, Gelatin, Mannitol, Citric acid, Chitosan, Sodium Alignate, Cyclodextrin, Hyaluronic acid and any combination thereof.

It is also within the scope of the present invention that the NSAID vaginal composition further comprises a probiotic component, such as probiotic bacteria, enzymes, vitamins and cellular factors.

A vaginal tablet with effervescent properties may comprise the following ingredients:

| | | |
|---|---|---|
| NSAID compound | 50-300 | mg (i.e. Ibuprofen 100 mg) |
| Danazol (optionally) | 10-150 | mg |
| Carbopol 971 | 10 | mg |
| Starch 1500 | 168 | mg |
| Citric acid | 45.6 | mg |
| Sodium bicarbonate | 34.4 | mg |
| PVP k30 | 3.9 | mg |
| Magnesium stearate | 10 | mg |

Each carrier unit containing the vaginal composition may include the following ingredients specified in Table 4 below:

TABLE 4
Ingredients and preparation steps of the effervescent composition

| Amount | Ingredient (raw | | Preparation |
|---|---|---|---|
| mg/gr | material) | mg/unit | Part |
| 148.3 | Danazol API | 150.0 | I |
| | NSAID | 50-300 | Wet |
| | | (According to | Granulation |
| | | Table 1) | |
| 141.41 | Starch 1500 | 143.0 | |
| 49.44 | Kollidone CL | 50.0 | |
| 39.56 | PVP 30 cps | 40.0 | |
| 247.22 | Lactose 21A | 250.0 | |
| * | Ethanol, BP | 175.0 | |
| * | Purified Water, | 35.0 | |
| | USP | | |
| 9.89 | Tween 80 | 10.0 | |
| 19.78 | Syloid F244 | 20.0 | II |
| 56.37 | Adipic/Lactic | 57.0 | Direct |
| | acid | | Compression |
| 148.33 | Lactose 30G | 150.0 | |
| 14.83 | Mg Stearate | 15.0 | |
| 49.44 | Sodium | 50.0 | |
| | bicarbonate | | |
| 14.83 | Carbomer 934P | 15.0 | |
| 8.1 | Opadry II White | 10.0 | III |
| * | Purified Water, | 80.0 | Coating |
| | USP | | |
| 1000 | | 1010 | Total |

### Example 4

### Preparation of a Danazol Composition Formulated as a Thin Film Type or Mucoadhesive Type Delivery Formulation for Vaginal Application

Reference is now made to a vaginal composition formulated in an immediate release form, comprising NSAID and/or NSAID combined with danazol as an active ingredients and at least one excipient adapted for thin film type or mucoadhesive type delivery. The aforementioned vaginal composition may also contain at least one FDT type excipient. Such a composition administered vaginally is effective in treatment of endometriosis, adenomyosis, menorrhagia, and dysmenorrhea.

The at least one thin film type or mucoadhesive type delivery excipient may be selected from a group comprising hydroxypropyl methyl cellulose (HPMC) K4M, Carbopol 934, Gelatin, Mannitol, Citric acid, Chitosan, Sodium Alignate, Cyclodextrin, Hyaluronic acid and any combination thereof.

The aforementioned vaginal composition may further include excipients as listed in Examples 1, 2 and 3 above, particularly the excipients listed in Tables 2, 3 and 4.

It is also within the scope of the present invention that the NSAID vaginal composition further comprises a probiotic component, such as probiotic bacteria, enzymes, vitamins and cellular factors.

### Example 5

### Preparation of an “Add-on” to a Tampon Formulation for Vaginal Application

The present invention further provides an “add-on” to a tampon formulation comprising NSAID and/or danazol as the active ingredient for vaginal application. The application of the composition in such a configuration has several significant advantages. A vaginal formulation as disclosed herein above, comprising the at least one active ingredient, is added to a conventional tampon as a thin layer or film with immediate release properties. In a preferred configuration, the thin layer or film is formulated as a coated sheet that at least partially covers the outer surface of the tampon. Upon contact with the vaginal epithelium and/or menstrual fluid or other vaginal discharges, effective therapeutic doses of the at least one active ingredient (i.e. at least one NSAID compound and/or danazol), rapidly disintegrate into the target tissue or organ, namely the vaginal and uterine cavities. The disintegrated residuals containing the active ingredients are formulated with bioadhesive characteristics such that the disintegrated composition residuals are designed to adhere to the vaginal tissue, and have a therapeutic effect close to the disease site, particularly at a heavy menstruation condition.

Thus the “add-on” tampon configuration encompass both, activities of absorbing menstrual fluid and, at the same time, releasing effective amounts of a specific NSAID compound, or a combination of more than one NSAID compound or a combination of danazol and at least one NSAID compound, directly to the vaginal cavity, particularly, at a menstruation period, for treating endometriosis, adenomyosis, menorrhagia, dysmenorrheal and menstruation pains.

One example of a formulation and configuration as described above is a catamenial tampon designed to be inserted into a body cavity and subsequently be in intimate contact with the vaginal epithelium. Such a pessary inserted into the vagina, may be coated with an immediate release formulation as disclosed herein above. Alternatively, an absorbent article worn exterior to the body may be used.

The compositions may be applied to the absorbent article using conventional methods for applying a pharmaceutical composition to the desired absorbent article. For example, unitary tampons without separate wrappers, may be dipped directly into a liquid bath having the composite and then can be air dried, if necessary to remove any volatile solvents. For compressed tampons, impregnating any of its elements is best done before compressing. The compositions when incorporated on and/or into the tampon materials may be fugitive, loosely adhered, bound, or any combination thereof. As used herein the term “fugitive” means that the composition is capable of migrating through the tampon materials.

In a specific embodiment, a conventional applicator may be used, suitable for vaginal administration of the “add-on” tampon formulation.

In a further embodiment, a catamenial tampon comprising absorbent material; and at least one outer layer comprising therapeutic amount of the vaginal composition comprising at least one NSAID compound or a combination of at least one NSAID compound and danazol in an immediate release formulation, is disclosed. Such a formulation is effective in inhibiting or treating endometriosis, adenomyosis, menorrhagia, dysmenorrheal and menstruation pains.

The present embodiment further relates to a process for adding pharmaceutically active compounds to substrates. The present invention is particularly useful for substrates used in the manufacture of disposable absorbent articles, specifically suited for substrates used in the manufacture of tampons.

The substrates of the present invention include disposable absorbent articles. Such articles can include patches for topical or transdermal applications, nasal pads or tampons, diapers, incontinence products, sanitary protection products, body wipes, bedsheets and surgical gowns. Preferably, the substrates are vaginal tampons.

### Example 6

### Preparation of a Foam Formulation for Vaginal Application

In a certain aspect, the present invention provides a pharmaceutical composition for vaginal application in a form of a foam or mousse. The pharmaceutical composition contains at least one NSAID or at least one NSAID combined with danazol as therapeutic active ingredients and at least one inactive ingredient or excipient selected from the lists disclosed herein above. In this embodiment the pharmaceutical composition further comprises a pharmaceutically acceptable carrier comprising at least one dispersing agent that is a foam forming agent.

In certain embodiments the dispersing agent is a foam forming agent selected from a surfactant, a cholesteryl ester, a fatty acid, a phospholipid, a carbohydrate, a protein or any combination thereof.

In certain embodiments the surfactant is selected from a group comprising a natural ionic surfactant, a synthetic ionic surfactant, a natural non ionic surfactant, a synthetic non-ionic surfactant and a mixture thereof.

In certain embodiments the pharmaceutical composition is provided as an aerosol foam or mousse and the composition further comprises a propellant. The propellant used may be chosen from conventional aerosol propellants. Thus, the propellant may be selected from propane, butane, dichloro difluoro methane, dichloro tetrafluoro ethane, octafluoro cyclobutane, and mixtures of two or more thereof. The propellant may be present in amounts of about 3 to about 30% w/w.

In other embodiments the composition is provided as non-aerosol foam or mousse.

In a further embodiment, the vaginal pharmaceutical composition is provided with a dispensing device adapted for the dispensing the pharmaceutical agent in a form of a foam or mousse.

Preferably, the medicament is formulated as a foamable composition, which, upon admixing with a gas propellant in an aerosol container, produces a foamable composition that is suitable for administration to the vaginal cavity. Alternatively the foamable composition is administered using a non-aerosol dispenser.

### Example 7

### Preparation Process of the Vaginally Administrable Composition

Reference is now made to a preparation procedure for formulating the vaginal composition comprising at least one NSAID as an active ingredient. Alternatively such a process is used herein to produce the danazol combined with at least one NSAID vaginal composition. Examples of NSAID compounds may include aspirin, ibuprofen, naproxen, Diclofenac, Cox-2 inhibitors, mefenamic acid, etodolac, indomethacin, ketoprofen, piroxicam, folmetin, diclofenac, piroxicam, tenoxicam, mecoxicam, meloxicam, ibufenac, ketoprofen, a pharmaceutically acceptable salt thereof and any combination thereof. The aforementioned procedure mainly includes three parts: (I) a wet granulation step, (II) a direct compression or compaction step or dry process, and optionally, (III) a coating step. More specifically, the preparation process may include the following steps:
a. Step 1: The active ingredient and excipients are weighed and mixed.
b. Step 2: The wet granulate is prepared by adding the liquid binder—adhesive and disintegrant to the powder blend and mixing thoroughly. Examples of binders/adhesives include aqueous preparations of starch, natural gums such as acacia, cellulose derivatives, such as methyl cellulose, gelatin, and povidone.
c. Step 3: Screening the damp mass through a mesh to form pellets or granules.
d. Step 4: Drying the granulation. A conventional tray-dryer or fluid-bed dryer are most commonly used.
e. Step 5: After the granules are dried, they are passed through a screen of smaller size than the one used for the wet mass to create granules of uniform size.

The wet granulation process may include the following steps:
(a) mixing gently, using a chopper, the active ingredient (NSAID and/or danazol) with the inactive matrix ingredients listed above, for about 5 minutes in high shear granulator (Diosna 1-6);
(b) adding water and granulating in high shear using impeller and chopper for about 2 minutes;
(c) drying in fluid bed for about 30 minutes in 60° C. inlet temp;
(d) screening through 1 mm sieve; and,
(e) adding Mg stearate and mixing for about 3 minutes.

An example of a process for the preparation of the aforesaid compositions may include the following procedure:

Equipment that may be used to prepare the vaginal composition comprise: Balance (ID-484); Diosna 1 L (ID-490); FBD Dryer (ID-727); Erweka (ID-492); Bin 1 L; Piccola (ID-481).

It is herein noted that the final blend procedure is performed after weighing the above ingredients.

The preparation steps below describe a preferred process for formulating the NSAID, or NSAID combined with danazol in a carrier unit administered vaginally.
1. All the ingredients are weighed and preferably preserved in laminate packages.
2. The ingredients of Part I (see Table 1 or 2 above) are mixed in a high shear granulator. High shear wet granulation processes use equipment that mixes the powder and liquid at a very fast rate, and thus speeds up the manufacturing process. The high shear granulation process is performed in the following conditions:
   a. Premix: The ingredients are premixed for 3 min in Rate (rpm): Impeller-200, Chopper-300.
   b. High shear granulation: Rate (rpm): Impeller-800, Chopper-1500. The ingredients are mixed with Ethanol solution and transferred to FBD.
   c. Drying: Part I materials are mixed manually and transferred into a glass vessel. The drying time is about 1.0 h at 50° C.
   d. Milling: The ingredients are milled with Erweka 0.6-1.0 mm and then sieved through 0.6 mm screen sieve manually to produce mixture I that is transferred into a PP vessel #1.
3. Part II materials are transferred into a glass vessel #2 to produce mixture II.
4. The Mg Stearate of Part II is sieved through screen sieve #45 manually and transferred into PP vessel #3.
5. Mixture I and mixture II are mixed together using 1 L Bin & Tumbler for 20 min at the speed of 12 rpm.
6. Mg Stearate (vessel #3) is added to the above mixture and mixed together for 5 min at the speed of 12 rpm.

### Example 8

### Preparation Process of a Vaginally Administrable Composition with Thin Film Type or Mucoadhesive Type Delivery Form

The preparation steps below describe a preferred process for formulating the at least one NSAID, danazol or danazol combined with at least one NSAID carriers of the present invention. In this example the carriers are formulated in a thin film type or mucoadhesive type delivery form. Non-limiting examples of thin film type or mucoadhesive type delivery excipients included within the scope of the present invention comprise hydroxypropyl methyl cellulose (HPMC) K4M, Carbopol 934, Gelatin, Mannitol, Citric acid, hyaluronic acid, pharmaceutically acceptable salts thereof and any combination thereof.

Non limiting examples of NSAID ingredient may include diclofenac, piroxicam, tenoxicam, mecoxicam, meloxicam, ibufenac, ibuprofen, naproxen, mefenamic acid and ketoprofen, pharmaceutically acceptable salts thereof and any mixture thereof.

### Preparation of a Polymeric Solution:

Thin films or strips or shreds comprising the compositions of the present invention are preferably prepared by a solvent casting method.
1. Weighing the required quantity of Hydroxyl propyl methyl cellulose (HPMC) and Carbopol to make 25 ml of a polymeric solution.
2. Adding ethanol and dichloromethane solvent to HPMC polymer to make the polymeric solution.
3. Adding calculated amount of glycerine and mixing properly.
4. Finally adding the Carbopol solution in water, after neutralization, to the above polymeric solution and mixing well.
5. Leaving the polymeric solution overnight at room temperature to obtain bubble free solution.

### Incorporation of the Drug:

1. Adding to the above solution, calculated amount of at least one NSAID, danazol or a combination thereof (4 mg/1.766 cm2) and mixing properly.
2. Keeping Petri dishes on surface level which is adjusted by spirit level.
3. Casting the polymeric solution onto Petri dishes and covering with glass funnel for controlling evaporation of ethanol and allowing it to dry at room temperature (25° C.) up to 30 hrs.
4. Cutting the dried film (1.5 cm diameter) and packing in an aluminum foil. Then storing in desiccator conditions until use.

Blank film is prepared following the same procedure, without addition of the drug.

It is herein emphasized that each of the above described steps may be performed by any conventional method known in the art.

While a number of exemplary aspects and embodiments have been discussed above, those who skilled in the art will recognize certain modifications, permutations, additions, and sub-combinations thereof. It is therefore intended that the following appended claims hereafter introduced be interpreted to include all such modifications, permutations, additions and sub-combinations as are within their true spirit and scope.

1-124. (canceled)

125. A pharmaceutical composition useful for treating at least one gynecological condition selected from the group consisting of: dysmenorrhea, mennorhagia and menstruation pains, said composition comprises at least one non-steroidal anti-inflammatory drug (NSAID) and at least one pharmaceutically acceptable carrier or excipient, wherein said composition is effervescent and in an immediate release disintegrating tablet form, adapted to be vaginally administrable.

126. The pharmaceutical composition of claim 125, wherein said composition is adapted to reduce menstrual blood loss by at least about 10%.

127. The pharmaceutical composition of claim 125, wherein said disintegrating tablet form is selected from the group consisting of a caplet, a capsule, a lozenge, a dragee, a wafer, a tab, a bar, a stick, a granule, a bead, a layer, a strip, a shred, a film, a Thin Film type, a PharmFilm type, a mucoadhesive type, a particle, nanoparticles, extrusion/spheronization particles, a matrix, a fast dissolving tablet (FDT) type, micronized particles, powder form, and any combination thereof.

128. The pharmaceutical composition of claim 125, wherein said carrier or excipient is selected from a group consisting of diluents, binders, granulating agents, glidants, lubricants, surfactants, disintegrants, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents and any combination thereof.

129. The pharmaceutical composition of claim 125, wherein said composition comprises between about 5 to about 12 wt. % effervescent excipient.

130. The pharmaceutical composition of claim 128, wherein at least one of the following holds true: (a) said at least one Thin Film type or PharmFilm type or mucoadhesive type excipient is selected from a group consisting of: hydroxypropyl methyl cellulose (HPMC) K4M, Carbopol 934, hyaluronic acid, Gelatin, Mannitol, Citric acid, Chitosan, Sodium Alignate, Cyclodextrin and combination thereof (b) said at least one FDT type excipient is selected from a group consisting of: Mannitol, Lactose, Erythritol, Xylitol, Glucose, Sucrose, Sorbitol, Maltitol, Trehalose, Maltose and mixtures thereof (c) said bioadhesive type agent is selected from a group consisting of Carbopol 971, hyaluronic acid, HPMC, hydrophilic polymers such as polyox, PEG (Polyethylene glycol), hydrogels, cellulose derivatives such as, sodium alginate, carboxymethylcellulose, hydroxymethylcellulose, methylcellulose, hydroxypropyl methyl cellulose (HPMC) K4M, Carbopol 934, Gelatin, Mannitol, Citric acid, Chitosan, Sodium Alignate, Cyclodextrin and any mixture thereof (d) said probiotic agent is selected from a group consisting of lactic-acid bacteria such as Lactobacillus rhamnosus, bifidobacterium, streptococcus, factors that stimulate the growth of protective organisms, enzymes, vitamins and cellular factors and mixtures thereof (e) said acidifying agent is selected from a group consisting of boric acid, acetic acid, glacial acetic acid, fumaric acid, diluted hydrochloric acid, citric acid, lactic acid, malic acid, nitric acid, phosphoric acid, sulfuric acid, tartaric acid, acidic buffer and mild organic acids approved for pharmaceutical applications and any combination thereof (f) said surfactant is selected from a group consisting of: a hydrophilic, a non hydrophilic, a polymeric, an ionic, a non ionic and zwitterionic surfactant, a natural ionic surfactant, a synthetic ionic surfactant, a natural non-ionic surfactant, a synthetic non-ionic surfactant and a mixture thereof (g) said protective agent is selected from a group consisting of antibacterial agents, antifungal agents, antiviral agents, agents capable of enhancing protection against sexually transmitted diseases, vitamins, minerals, enzymes, co-enzymes, co-factors, microorganisms, organic acids, probiotic bacteria, and a variety of molecules extracted from natural sources such as amino acids, polysaccharides, peptides, naturally occurring hormones, biochemical intermediates, and any combination thereof and (h) said dispersing agent is selected from a group consisting of a surfactant, a cholesteryl ester, a fatty acid, a phospholipid, a carbohydrate, a protein and any mixture thereof.

131. The pharmaceutical composition of claim 125, wherein said composition has disintegration time of between about 3 minutes and about 60 minutes in the vagina.

132. The pharmaceutical composition of claim 125, wherein said composition has a disintegration time of less than about 3 minutes in water at room temperature, or between about 3 minutes and about 15 minutes in water at room temperature.

133. The pharmaceutical composition according to claim 125, wherein said at least one NSAID is selected from a group consisting of: Salicylates, Propionic acid derivatives, Acetic acid derivatives, Enolic acid (Oxicam) derivatives, Fenamic acid derivatives (Fenamates), Selective COX-2 inhibitors (Coxibs), Sulphonanilides, prostaglandin synthetase inhibitors, aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, and a pharmaceutically acceptable salt or derivative thereof and any combination thereof.

134. The pharmaceutical composition of claim 125, wherein at least one of the following holds true: (a) said at least one NSAID is configured for administration in dosage unit of between about 50 mg to about 300 mg; (b) said at least one NSAID is configured for administration in a daily dosage of between about 5 mg to about 2000 mg; and (c) said composition is configured for administration vaginally one to six times per day.

135. The pharmaceutical composition of claim 133, wherein at least one of the following holds true: (a) said Diclofenac is configured for administration in a dosage unit of between about 10 mg to about 50 mg, two times per day, or in a daily dosage unit of between about 10 mg to about 100 mg; (b) said Naproxen is configured for administration in a dosage unit of between about 25 mg to about 500 mg, one to two times per day; (c) said Piroxicam is configured for administration in a daily dosage unit of between about 5 mg to about 20 mg; (d) said Ibuprofen is configured for administration in a dosage unit of between about 20 mg to about 200 mg, one to three times per day; (e) said Indomethacin is configured for administration in a dosage unit of between about 5 mg to about 25 mg, one to six times per day; (f) said Mefenamic acid is configured for administration in a dosage unit of between about 25 mg to about 500 mg, one to four times per day.

136. The pharmaceutical composition of claim 125, wherein said composition has a rapid disintegration time in the vagina.

137. The pharmaceutical composition of claim 125, wherein at least one of the following holds true: (a) said at least one NSAID has a lower absolute or systemic bioavailability ratio in the serum or blood stream as compared to conventional oral NSAID compositions comprising comparable amounts of said at least one NSAID; (b) said at least one NSAID has a higher relative bioavailability ratio at the endometrial and/or vaginal tissue as compared to conventional oral NSAID compositions comprising comparable amounts of said at least one NSAID.

138. The pharmaceutical composition of claim 125, wherein said composition is characterized by a property selected from a group consisting of enhanced adhesiveness to the endometrial and/or vaginal tissue, enhanced solubility in the endometrial and/or vaginal tissue, enhanced disintegration or dissolving rate at the endometrial and/or vaginal tissue, enhanced bioavailability, improved drug release ratio, improved mucoadhesive strength and any combination thereof, relative to conventional oral compositions comprising comparable amounts of said at least one NSAID.

139. The pharmaceutical composition of claim 125, wherein said composition confers at least one synergistic effect with respect to treatment of a gynecological condition selected from a group consisting of menorrhagia, dysmenorrheal, menstruation pain and any combination thereof by having more than an additive effect relative to the effect conferred by conventional oral compositions comprising comparable amounts of said at least one NSAID.

140. A pharmaceutical composition for vaginal administration, prepared by steps of:
a. preparing a mixture comprising (i) an effective amount of at least one NSAID, and (ii) at least one pharmaceutically acceptable carrier or excipient and,
b. forming said composition in an immediate release effervescent disintegrating tablet form.

141. The composition for vaginal administration prepared by steps according claim 140, wherein said mixture is further prepared by steps of:
a. adding water to said mixture to prepare a second mixture;
b. granulating said second mixture;
c. adding an effervescent excipient to said second mixture,
d. drying said granulation mixture; and,
e. compressing said granules so as to form said disintegrating effervescent tablet form.

142. A method for treating a gynecological condition, especially dysmenorrhea, mennorhagia and menstruation pains, wherein said method comprises steps of:
a. formulating a composition comprising at least one non-steroidal anti-inflammatory drug (NSAID) and at least one carrier or excipient;
b. administering said composition vaginally at a therapeutically effective dosage;
wherein said method further comprises steps of formulating said composition in an effervescent immediate release disintegrating tablet form.

143. The method of claim 142, wherein said method further comprises steps of formulating said composition with between about 5 to about 12 wt. % effervescent excipient.

144. The method of claim 142, comprising an additional step of conferring at least one synergistic effect with respect to treatment of a gynecological condition selected from a group consisting of menorrhagia, dysmenorrheal, menstruation pain and any combination thereof by having more than an additive effect relative to the effect conferred when comparable amounts of said at least one NSAID is administered orally.
